(19) United States
(12) Patent Application Publication
(10) Pub. No.: US 2007/0293542 A1
(43) Pub. Date: Dec. 20, 2007

(54) Selective Cox-2 Inhibitors

(76) Inventors: Brian M. Cali, Arlington, MA (US); Yueh-Tyng Chien, Newton, MA (US); Mark G. Currie, Sterling, MA (US); John Jeffrey Talley, Somerville, MA (US); Craig Zimmerman, Topsfield, MA (US)

Correspondence Address: FISH & RICHARDSON PC, P.O. BOX 1022, MINNEAPOLIS, MN, 55440-1022, US

(21) Appl. No.: 10/575,939

(22) PCT Filed: Oct. 18, 2004
(86) PCT No.: PCT/US04/34385

§ 371 Date: Jan. 29, 2007

Related U.S. Application Data
(60) Provisional application No. 60/511,792, filed on Oct. 16, 2003.

(51) Int. Cl. A61K 31/444; A61K 31/341; A61K 31/4152; A61K 31/42; A61P 25/22; C07D 213/22; C07D 261/06; C07D 275/02; C07D 307/32; C07D 277/20; C07D 263/30; C07D 231/00; A61P 29/00; A61K 31/421; A61K 31/425; A61K 31/426

(52) U.S. Cl. 514/334; 514/365; 514/372; 514/374; 514/378; 514/404; 514/473; 546/258; 548/204; 548/214; 548/235; 548/247; 548/366.4; 549/479

(57) ABSTRACT

The invention features compounds that are inhibitors of COX-2 activity. Certain of the compounds are inhibitors of FAAH activity as well as COX-2 activity. The compounds include a 5-membered or 6-membered substituted or unsubstituted heteroaryl or heterocyclyl ring having one or two heteroatoms selected from the group consisting of O, S, and N that is linked to two substituted or unsubstituted 6-membered aryl or heteroaryl groups that can be the same or different.

### RELATED APPLICATION INFORMATION

This application claims priority to U.S. provisional application Ser. No. 60/511,792, filed Oct. 16, 2003, hereby incorporated by reference.

### TECHNICAL FIELD

This invention relates to inhibitors of cyclooxygenase and inhibitors of fatty acid amide hydrolase.

### BACKGROUND

Cyclooxygenases play an essential role in prostaglandin synthesis. Cyclooxygenase-1 (COX-1) is constitutive and relatively long-lived, whereas cyclooxygenase-2 (COX-2) is inducible and relatively short-lived. COX-1 is thought to be responsible for maintaining basal level prostaglandin production, which is important for normal gastrointestinal and renal function. COX-2 is induced by certain inflammatory agents, hormones, growth factors, cytokines, and other agents. COX-2 plays a significant role in prostaglandin synthesis within inflammatory cells such as macrophages and monocytes, and prostaglandin production associated with COX-2 induction can have a deleterious effect on the body. Thus, to reduce unwanted pain and inflammation and to treat certain other conditions, while retaining normal gastrointestinal function, it can be desirable to inhibit COX-2 activity without inhibiting COX-1 activity.

Many non-steroidal anti-inflammatory drugs (NSAIDs) inhibit both COX-1 and COX-2. These non-selective inhibitors include indomethacin (Shen et al. 1963 J. Am. Chem. Soc. 85:4881; 4-chlorobenzoyl-5-methoxy-2-methyl-1H-indole-3-acetic acid). It is desirable to identify NSAIDs that are selective inhibitors of COX-2 activity, but do not significantly inhibit COX-1 activity at physiological levels where COX-2 activity is significantly inhibited. Such selective inhibitors are expected to have the desirable anti-inflammatory, anti-pyretic, analgesic properties associated with NSAIDs, while having reduced or no gastrointestinal and/or renal toxicity.

Subsequent to indomethacin administration, the unchanged parent compound, the desmethyl metabolite (O-desmethylindomethacin), the desbenzoyl metabolite (N-deschlorobenzoylindomethacin) and the desmethyl-desbenzoyl metabolite (O-desmethyl-N-deschlorobenzoylindomethacin) can be detected in plasma (Strachman et al. 1964 J. Am Chem. Soc. 8:799; Helleberg 1981 Clin Pharmacokinet. 6:245), all in an unconjugated form (Harman et al. 1964 J. Pharmocol Exp Therap 143:215). It has been reported that all three metabolites are devoid of anti-inflammatory activity (Helleberg 1981 Clin Pharmacokinet. 6:245 and Duggan et al. 1972 J. Pharmacol. and Exp. Ther. 181:562), although it has also been reported that the desmethyl metabolite has some ability to inhibit prostaglandin synthesis (Shen and Winter 1977 Adv Drug Res. 12:90).

Indomethacin derivatives in which the benzoyl group has been replaced by a 4-bromobenzyl group or the acetic acid side chain has been extended exhibit greater selectivity for inhibition of COX-2 relative to COX-1 (Black et al. 1996 Bioorganic & Medicinal Chem. Lett. 6:725 and Black et al. 1997 Advances in Experimental Medicine and Biology 407:73). In addition, synthesis methodology has been demonstrated for the preparation of indomethacin analogues, some of which do not inhibit cyclooxygenases (Touhey et al. 2002 Eur J Cancer 38:1661).

Many COX inhibitors, including indomethacin, are analgesics. However, a number of relatively selective COX-2 inhibitors exhibit rather slow onset of analgesic activity. It is thought that the rather poor aqueous solubility and bioavailability of many COX-2 inhibitors, as well as their tendency to bind plasma proteins account, at least in part, for the observed slow onset of analgesic activity. Many patients treated for pain with COX-2 inhibitors experience breakthrough pain. This can lead to the use of a second, sometimes less desirable, analgesic or higher doses of the COX-2 inhibitor (i.e., “dose creep”).

In some respects indomethacin seems to be a better analgesic than other COX-2 inhibitors (Goodman and Gilman, The Pharmacological Basis of Therapeutics. 8^{th} Edition, 1993, McGraw-Hill, Inc. New York, pp. 659-661).

Many fatty acid amides are known to have analgesic activity. A number of fatty acid amides (e.g., arachidonyl amino acids and anandamide) induce analgesia in animal models of pain (see, for example, Walker et al. 1999 Proc Natl Acad Sci 96:12198, Fride and Mechoulam 1993 Eur J Pharmacol 231:313). Anandamide and certain other fatty acid amides (e.g., N-palmitoyl ethanolamine, N-oleoyl ethanolamide, oleamide, 2-arachidonoylglycerol) are cleaved and inactivated by fatty acid amide hydrolase (FAAH) (Deutsch et al. 2003 Prostaglandins Leukot Essent Fatty Acids 66:201; and Cravatt and Lichtman 2003 Current Opinion in Chemical Biology 7:469).

Inhibition of FAAH is expected to lead to an increase in the level of anandamide and other fatty acid amides. This increase in fatty acid amides may lead to an increase in the nociceptive threshold. Thus, inhibitors of FAAH are useful in the treatment of pain. Such inhibitors might also be useful in the treatment of other disorders that can be treated using fatty acid amides or modulators of cannabinoid receptors (e.g., anxiety, eating disorders, and cardiovascular disorders). NPAA (N-palmitoylethanolamine acid anhydrolase) is a hydrolase that breaks down N-palmitoyl ethanolamine (PEA), a fatty acid amide. PEA is a naturally occurring substrate for the cannabinoid receptor 2 (CB2 receptor). Inhibition of NPAA may lead to increased PEA levels. Accordingly, NPAA inhibitors may be useful in the treatment of inflammation and nociceptive pain control.

In addition, there is evidence (see, e.g., Weber et al. 2004 J. Lipid Res. 45:757) that when FAAH activity is reduced or absent, one of its substrates, anandamide, acts as a substrate for COX-2 that can be converted to a prostamide. Thus, certain prostamides may be elevated in the presence of an FAAH inhibitor. Given that certain prostamides are associated with reduced intraocular pressure and ocular hypotensivity, FAAH inhibitors may be useful agents for treating glaucoma.

### SUMMARY

The invention features compounds having Formula I or Formula II or Formula IIa or a pharmaceutically acceptable salt thereof, pharmaceutical compositions comprising such compounds and methods for treating a patient by administering such pharmaceutical compositions alone or in combination with one or more other therapeutic agents.

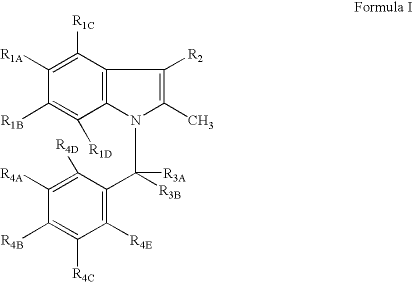

Thus, the invention features a compound having Formula I, wherein:
R_{1A} is selected from: a prodrug of a hydroxy moiety, a hydroxy group, —OC(O)CH₃, an ester having a C₁₋₆ branched or straight chain alkyl group, phosphate ester having C₁₋₆ branched or straight alkyl groups, a carbamate having C₁₋₆ branched or straight alkyl groups, and a carbonate group having a C₁₋₆ branched or straight chain alkyl group.
R_{1B} and R_{1C} and R_{1D} are independently selected from: H, CH₃ or a halogen or subset of halogens (F, Cl, Br or F, Cl, Br, and I);
R₂ is selected from A, B, and C, wherein: 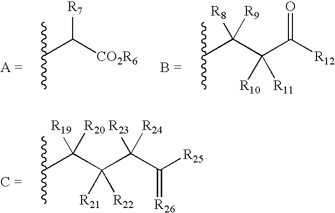
R_{3A} and R_{3B} are both H or together form an oxo group; and
R_{4A}, R_{4B}, R_{4C}, R_{4D} and R_{4E} are independently selected from:
   (a) H,
   (b) F, Cl, Br, and I,
   (c) C₁₋₆ alkyl, and
   (d) —SCH₃, —S(O)CH₃, SCH₂CH₃, —SCF₂H or —SCF₃.
   (e) OCF₃, OCF₂H

For compounds having Formula I in which R₂ is A, R₆ is H or C₁₋₆ alkyl, in some embodiments C₁₋₆ alkyl and R₇ is selected from: H, C₁₋₆ alkyl C₁₋₃ alkyl, and is preferably selected from H, methyl, and ethyl. In certain embodiments R_{1D} is H. In certain embodiments R_{4A}, R_{4C}, R_{4D} and R_{4E} are H.

Among the embodiments in which R₂ is A, useful compounds include: 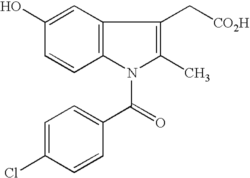
[1-(4-chlorobenzoyl)-5-hydroxy-2-methyl-1H-indol-3-yl]acetic acid, 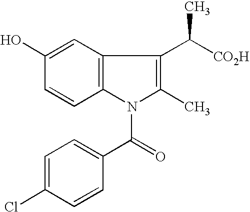
(2R)-2-[1-(4-chlorobenzoyl)-5-hydroxy-2-methyl-1H-indol-3-yl]propionic acid 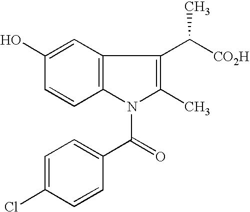
(2S)-2-[1-(4-chlorobenzoyl)-5-hydroxy-2-methyl-1H-indol-3-yl]propionic acid 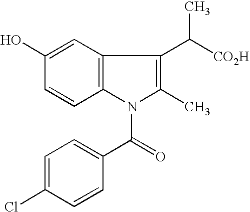
2-[1-(4-chlorobenzoyl)-5-hydroxy-2-methyl-1H-indol-3-yl]propionic acid. 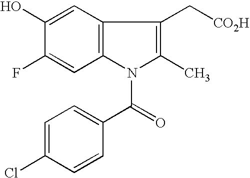
[1-(4-chlorobenzoyl)-fluoro-5-hydroxy-2-methyl-1H-indol-3-yl]acetic acid 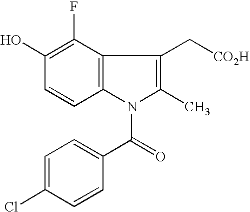
[1-(4-chlorobenzoyl)-5-fluoro-5-hydroxy-2-methyl-1H-indol-3-yl]acetic acid 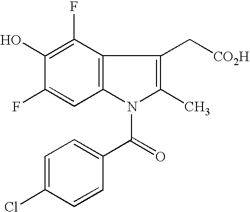
[1-(4-chlorobenzoyl)-4,6-difluoro-5-hydroxy-2-methyl-1H-indol-3-yl]acetic acid 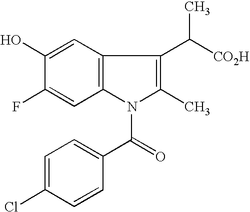
2-[1-(4-chlorobenzoyl)-6-fluoro-5-hydroxy-2-methyl-1H-indol-3-yl]propionic acid 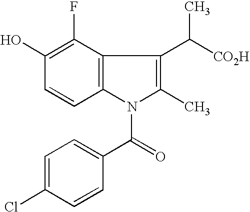
2-[1-(4-chlorobenzoyl)-5-fluoro-5-hydroxy-2-methyl-1H-indol-3-yl]propionic acid 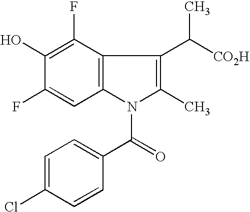
2-[1-(4-chlorobenzoyl)-4,6-difluoro-5-hydroxy-2-methyl-1H-indol-3-yl]propionic acid 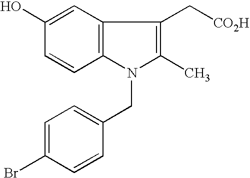
[1-(4-bromobenzyl)-5-hydroxy-2-methyl-1H-indol-3-yl]acetic acid 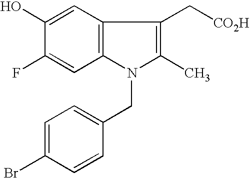
[1-(4-bromobenzyl)-6-fluoro-5-hydroxy-2-methyl-1H-indol-3-yl]acetic acid 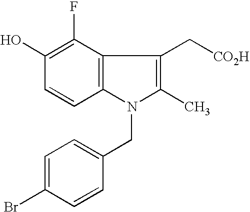
[1-(4-bromobenzyl)-4-fluoro-5-hydroxy-2-methyl-1H-indol-3-yl]acetic acid 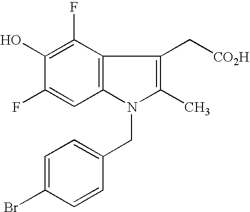
[1-(4-bromobenzyl)-4,6-fluoro-5-hydroxy-2-methyl-1H-indol-3-yl]acetic acid 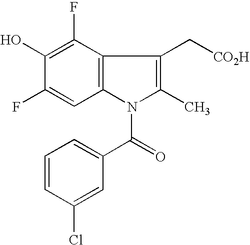
[1-(3-chlorobenzoyl)-4,6-difluoro-5-hydroxy-2-methyl-1H-indol-3-yl]acetic acid 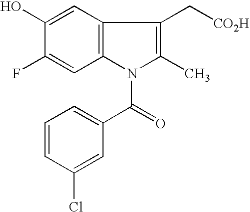
[1-(3-chlorobenzoyl)-6-fluoro-5-hydroxy-2-methyl-1H-indol-3-yl]acetic acid 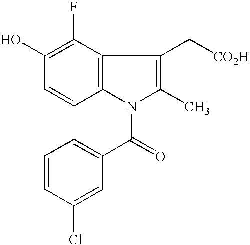
[1-(3-chlorobenzoyl)-4-fluoro-5-hydroxy-2-methyl-1H-indol-3-yl]acetic acid 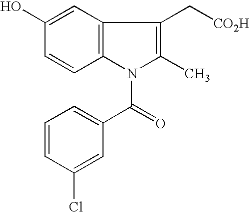
[1-(3-chlorobenzoyl)-5-hydroxy-2-methyl-1H-indol-3-yl]acetic acid 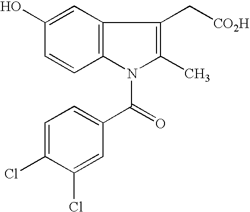
[1-(3,4-dichlorobenzoyl)-5-hydroxy-2-methyl-1H-indol-3-yl]acetic acid 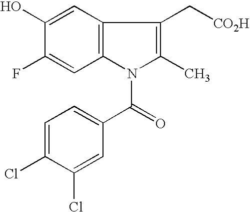
[1-(3,4-dichlorobenzoyl)-6-fluoro-5-hydroxy-2-methyl-1H-indol-3-yl]acetic acid 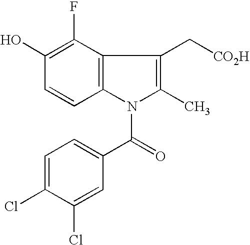
[1-(3,4-dichlorobenzoyl)-4-fluoro-5-hydroxy-2-methyl-1H-indol-3-yl]acetic acid 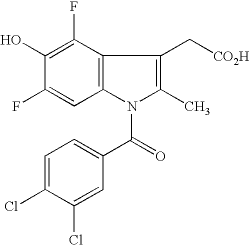
[1-(3,4-dichlorobenzoyl)-4,6-difluoro-5-hydroxy-2-methyl-1H-indol-3-yl]acetic acid 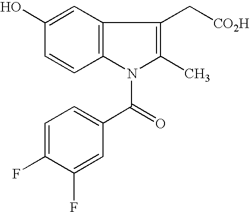
[1-(3,4-difluorobenzoyl)-5-hydroxy-2-methyl-1H-indol-3-yl]acetic acid 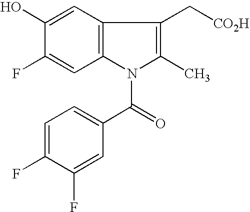
[1-(3,4-difluorobenzoyl)-6-fluoro-5-hydroxy-2-methyl-1H-indol-3-yl]acetic acid 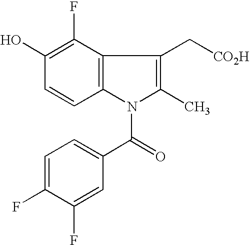
[1-(3,4-difluorobenzoyl)-4-fluoro-5-hydroxy-2-methyl-1H-indol-3-yl]acetic acid 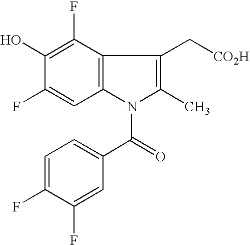
[1-(3,4-difluorobenzoyl)-4,6-difluoro-5-hydroxy-2-methyl-1H-indol-3-yl]acetic acid 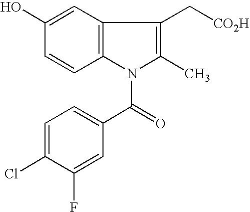
[1-(4-chloro-3-fluorobenzoyl)-5-hydroxy-2-methyl-1H-indol-3-yl]acetic acid 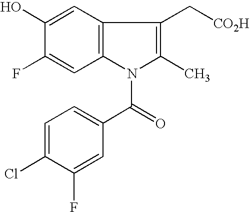
[1-(4-chloro-3-fluorobenzoyl)-6-fluoro-5-hydroxy-2-methyl-1H-indol-3-yl]acetic acid 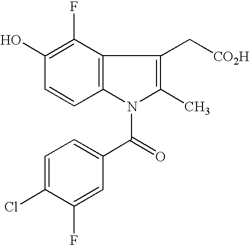
[1-(4-chloro-3-fluorobenzoyl)-4-fluoro-5-hydroxy-2-methyl-1H-indol-3-yl]acetic acid 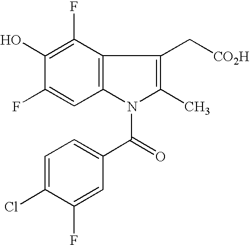
[1-(4-chloro-3-fluorobenzyl)-4,6-difluoro-5-hydroxy-2-methyl-1H-indol-3-yl]acetic acid 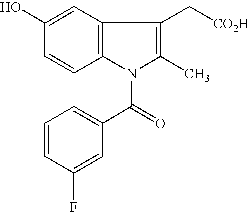
[1-(3-fluorobenzoyl)-5-hydroxy-2-methyl-1H-indol-3-yl]acetic acid 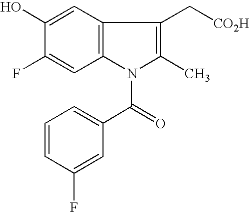
[6-fluoro-1-(3-fluorobenzoyl)-5-hydroxy-2-methyl-1H-indol-3-yl]acetic acid 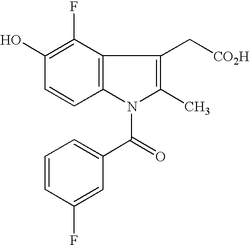
[4-fluoro-1-(3-fluorobenzoyl)-5-hydroxy-2-methyl-1H-indol-3-yl]acetic acid 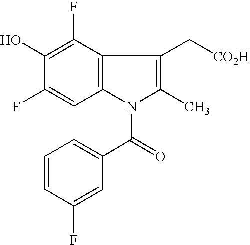
[4,6-difluoro-1-(3-fluorobenzoyl)-5-hydroxy-2-methyl-1H-indol-3-yl]acetic acid 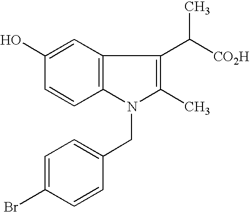
2-[1-(4-bromobenzyl)-5-hydroxy-2-methyl-1H-indol-3-yl]propionic acid 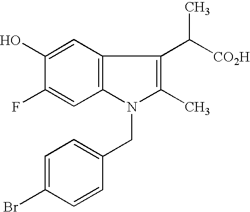
2-[1-(4-bromobenzyl)-6-fluoro-5-hydroxy-2-methyl-1H-indol-3-yl]propionic acid 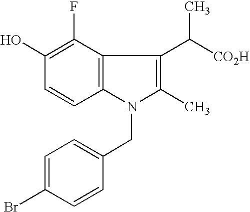
2-[1-(4-bromobenzyl)-4-fluoro-5-hydroxy-2-methyl-1H-indol-3-yl]propionic acid 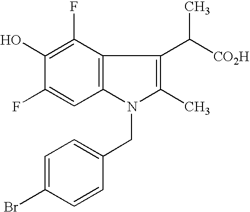
2-[1-(4-bromobenzyl)-4,6-difluoro-5-hydroxy-1H-indol-3-yl]propionic acid 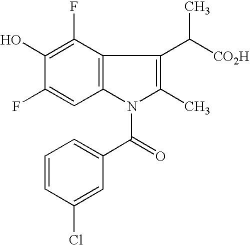
2-[1-(3-chlorobenzoyl)-4,6-difluoro-5-hydroxy-2-methyl-1H-indol-3-yl]propionic acid 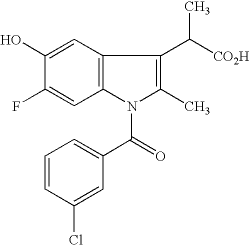
2-[1-(3-chlorobenzoyl)-6-fluoro-5-hydroxy-2-methyl-1H-indol-3-yl]propionic acid 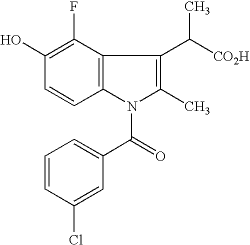
2-[1-(3-chlorobenzoyl)-4-fluoro-5-hydroxy-2-methyl-1H-indol-3-yl]propionic acid 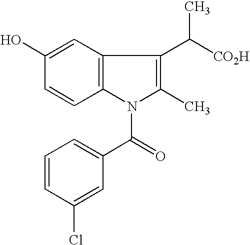
2-[1-(3-chlorobenzoyl)-5-hydroxy-2-methyl-1H-indol-3-yl]propionic acid 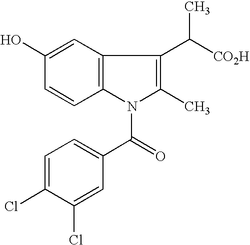
2-[1-(3-dichlorobenzoyl)-5-hydroxy-2-methyl-1H-indol-3-yl]propionic acid 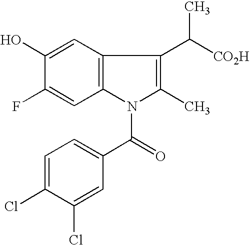
2-[1-(3,4-dichlorobenzoyl)-6-fluoro-5-hydroxy-2-methyl-1H-indol-3-yl]propionic acid 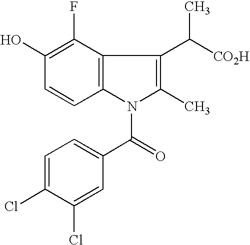
2-[1-(3,4-dichlorobenzoyl)-4-fluoro-5-hydroxy-2-methyl-1H-indol-3-yl]propionic acid 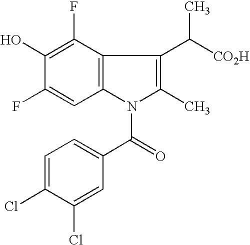
2-[1-(3,4-dichlorobenzoyl)-4,6-fluoro-5-hydroxy-2-methyl-1H-indol-3-yl]propionic acid 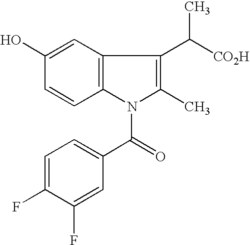
2-[1-(3,4-difluorobenzoyl)-5-hydroxy-2-methyl-1H-indol-3-yl]propionic acid 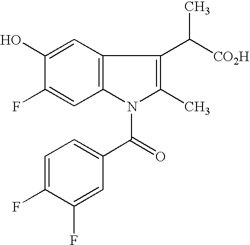
2-[1-(3,4-difluorobenzoyl)-6-fluoro-5-hydroxy-2-methyl-1H-indol-3-yl]propionic acid 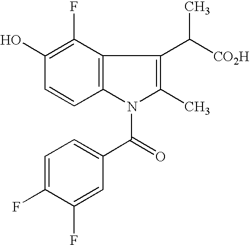
2-[1-(3,4-difluorobenzoyl)-4-fluoro-5-hydroxy-2-methyl-1H-indol-3-yl]propionic acid 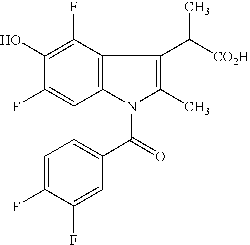
2-[1-(3,4-difluorobenzoyl)-4,6-difluoro-5-hydroxy-2-methyl-1H-indol-3-yl]propionic acid 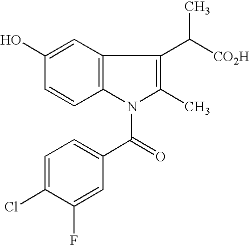
2-[1-(4-chloro-3-fluorobenzoyl)-5-hydroxy-2-methyl-1H-indol-3-yl]propionic acid 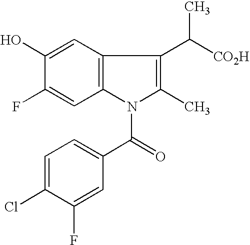
2-[1-(4-chloro-3-fluorobenzoyl)-6-fluoro-5-hydroxy-2-methyl-1H-indol-3-yl]propionic acid 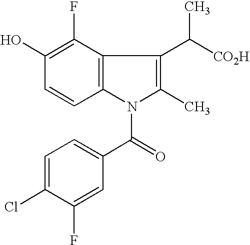
2-[1-(4-chloro-3-fluorobenzoyl)-4-fluoro-5-hydroxy-2-methyl-1H-indol-3-yl]propionic acid 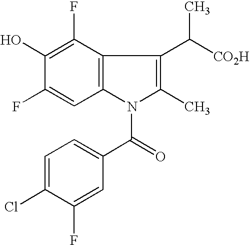
2-[1-(4-chloro-3-fluorobenzoyl)-4,6-difluoro-5-hydroxy-2-methyl-1H-indol-3-yl]propionic acid 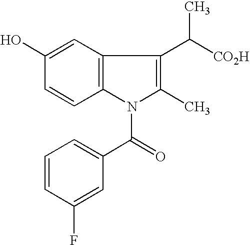
2-[1-(3-fluorobenzoyl)-5-hydroxy-2-methyl-1H-indol-3-yl]propionic acid 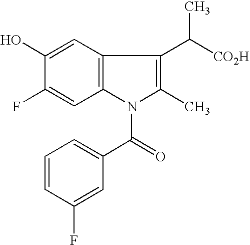
2-[6-fluoro-1-(3-fluorobenzoyl)-5-hydroxy-2-methyl-1H-indol-3-yl]propionic acid 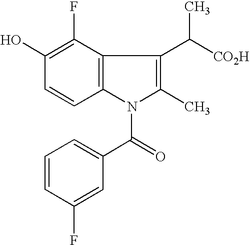
2-[4-fluoro-1-(3-fluorobenzoyl)-5-hydroxy-2-methyl-1H-indol-3-yl]propionic acid 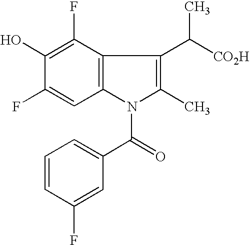
2-[4,6-difluoro-1-fluorobenzoyl)-5-hydroxy-2-methyl-1H-indol-3-yl]propionic acid 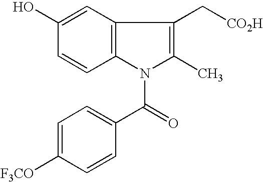
1-(4-trifluoromethoxybenzoyl)-5-hydroxy-2-methyl-1H-indol-3-yl-acetic acid 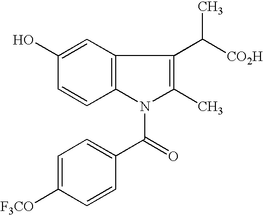
2-[1-(4-trifluoromethoxybenzoyl)-5-hydroxy-2-methyl-1H-indol-3-yl]propionic acid 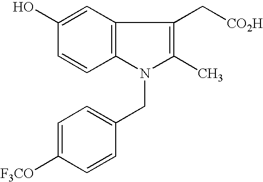
[1-(4-trifluoromethoxybenzyl)-5-hydroxy-2-methyl-1H-indol-3-yl]acetic acid 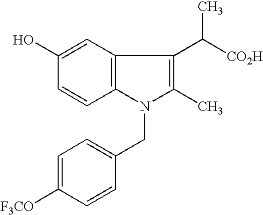
2-[1-(4-trifluoromethoxybenzyl)-5-hydroxy-2-methyl-1H-indol-3-yl]propionic acid 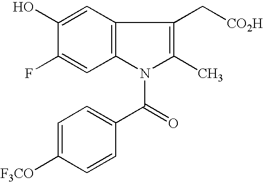
[1-(4-trifluoromethoxybenzoyl)-6-fluoro-5-hydroxy-2-methyl-1H-indol-3-yl]acetic acid 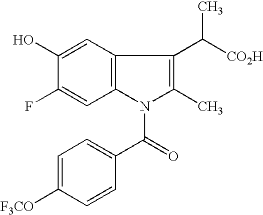
2-[1-(4-trifluoromethoxybenzoyl)-6-fluoro-5-hydroxy-2-methyl-1H-indol-3-yl]propionic acid 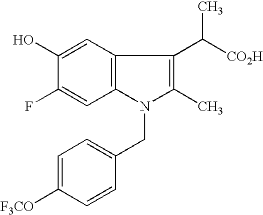
2-[1-(4-trifluoromethoxybenzyl)-6-fluoro-5-hydroxy-2-methyl-1H-indol-3-yl]propionic acid 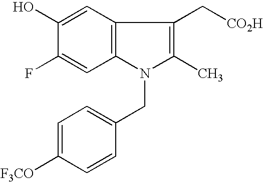
[1-(4-trifluoromethoxybenzyl)-6-fluoro-5-hydroxy-2-methyl-1H-indol-3-yl]acetic acid 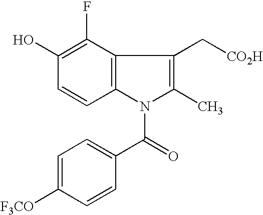
[1-(4-trifluoromethoxybenzoyl)-4-fluoro-5-hydroxy-2-methyl-1H-indol-3-yl]acetic acid 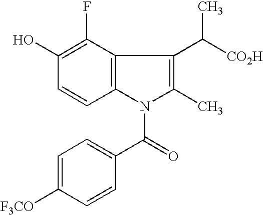
2-[1-(4-trifluoromethoxybenzoyl)-4-fluoro-5-hydroxy-2-methyl-1H-indol-3-yl]propionic acid 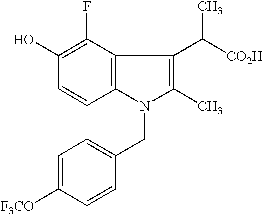
2-[1-(4-trifluoromethoxybenzyl)-4-fluoro-5-hydroxy-2-methyl-1H-indol-3-yl]propionic acid 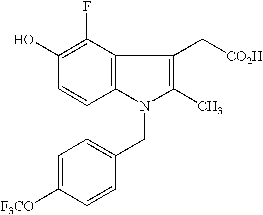
[1-(4-trifluoromethoxybenzyl)-4-fluoro-5-hydroxy-2-methyl-1H-indol-3-yl]acetic acid 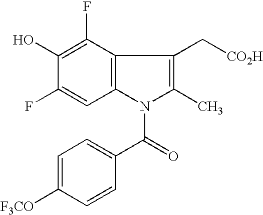
[1-(4-trifluoromethoxybenzoyl)-4,6-difluoro-5-hydroxy-2-methyl-1H-indol-3-yl]acetic acid 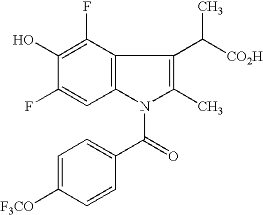
2-[1-(4-trifluoromethoxybenzoyl)-4,6-difluoro-5-hydroxy-2-methyl-1H-indol-3-yl]propionic acid 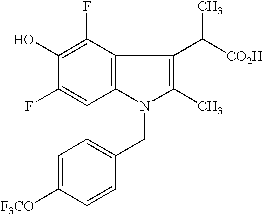
2-[1-(4-trifluoromethoxybenzyl)-4,6-difluoro-5-hydroxy-2-methyl-1H-indol-3-yl]propionic acid 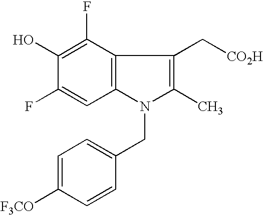
[1-(4-trifluoromethoxybenzyl)-4,6-difluoro-5-hydroxy-2-methyl-1H-indol-3-yl]acetic acid

Among compounds having Formula I wherein R₂ is B:
R₈, R₉, R₁₀, and R₁₁ are independently selected from:
   (a) H,
   (b) F,
   (c) methyl or ethyl,
   (d) —CF₃, —CF₂H or —CFH₂,
   (e) hydroxy, —OR₁₄, SR₁₄, S(O)R₁₄, or S(O)₂R₁₄, and
   (f) mono-substituted or di-substituted benzyl, wherein the substituents are selected from:
      (i) H,
      (ii) —CF₃,
      (iii) —CN,
      (iv) F, Cl, Br, or I,
      (v) C₁₋₆ alkyl, and
      (vi) SR₁₄, S(O)R₁₄, or S(O)₂R₁₄,
   (g) naphthylmethyl,
or R₈ and R₉ together form an oxo group,
or R₁₀ and R₁₁ together form an oxo group,
or R₈ and R₉ are joined so that together with the carbon atom to which they are attached there is formed a saturated 3, 4, 5, 6 or 7 membered hydrocarbon ring;
or R₈ and R₁₀ are joined so that together with the carbon atom to which they are attached there is formed a saturated 3, 4, 5, 6 or 7 membered hydrocarbon ring;
or R₈ and R₁₁ are joined so that together with the carbon atom to which they are attached there is formed a saturated 3, 4, 5, 6 or 7 membered hydrocarbon ring;
or R₉ and R₁₀ are joined so that together with the carbon atom to which they are attached there is formed a saturated 3, 4, 5, 6 or 7 membered hydrocarbon ring,
or R₉ and R₁₁ are joined so that together with the carbon atom to which they are attached there is formed a saturated 3, 4, 5, 6 or 7 membered hydrocarbon ring;
or R₁₀ and R₁₁ are joined so that together with the carbon atom to which they are attached there is formed a saturated 3, 4, 5, 6 or 7 membered hydrocarbon ring;
R₁₂ is selected from:
   (a) R₁₃, and
   (b) —NR₁₅R₁₆;
R₁₃ is selected from:
   (a) H, and
   (b) C₁₋₄ alkyl;
R₁₄ is selected from methyl, ethyl, mono-substituted benzyl or di-substituted benzyl, wherein the substituents are selected from:
   (a) H,
   (b) CF₃,
   (c) CN,
   (d) F, Cl, Br, I, and
   (e) C₁₋₆ alkyl;
R₁₅ and R₁₆ are independently selected from:
   (a) H,
   (b) C₁₋₃ alkyl,
   (c) —OR₁₃,
   (d) —C(O)R₁₇
   (e) —S(O)₂R₁₈,
   (f) a mono-substituted C₂₋₄ alkyl, wherein the substituent is selected firm:
      (i) hydroxy,
      (ii) amino,
      (iii) methylamino,
      (iv) dimethyl amino, and
   (g) an unsubstituted, mono-substituted, or disubstituted phenyl, benzyl or pyridyl group, wherein the substituents are selected from:
      (i) H,
      (ii) CF₃,
      (iii) CN,
      (iv) F, Cl, Br, I, and
      (v) C₁₋₆ alkyl;
or R₁₅ and R₁₆ are joined so that together with the nitrogen atom to which they are attached there is formed a 3, 4, 5, 6, or 7-membered ring, optionally including one or two additional heteroatoms, wherein the additional heteroatoms are selected from N, O, and S, the ring optionally including one or two carbonyl or sulfonyl groups;
R₁₇ is selected from:
   (a) H,
   (b) C₁₋₄ alkyl
   (c) CF₃, and
   (d) an unsubstituted, mono-substituted, or disubstituted phenyl or benzyl group, wherein the substituents are selected from:
      (i) H,
      (ii) CF₃,
      (iii) CN,
      (iv) F, Cl, Br, I, and
      (v) C₁₋₆ alkyl;
R₁₈ is selected from:
   (a) C₁₋₄ alkyl,
   (b) CF₃, and
   (c) an unsubstituted, mono-substituted, or disubstituted phenyl or benzyl group, wherein the substituents are selected from:
      (i) H,
      (ii) CF₃,
      (iii) CN,
      (iv) F, Cl, Br, I, and
      (V) C₁₋₆ alkyl.

Among compounds having Formula I wherein R₂ is C:
R₁₉, R₂₀, R₂₁, R₂, R₂₃ and R₂₄ are independently selected from:
   (a) H,
   (b) F or Cl,
   (c) C₁₋₅ alkyl or haloalkyl,
   (d) C₃₋₆ cycloalkyl,
   (e) —CF₃, —CF₂H or —CFH₂;
   (f) hydroxy, —OR₂₇, SR₂₇, S(O)R₂₇, or S(O)2R₂₇,
   (g) mono-substituted or di-substituted benzyl, wherein the substituents are selected from:
      (i) —CF₃,
      (ii) —CN,
      (iii) F, Cl, Br, or I,
      (iv) C₁₋₆ alkyl, and
      (v) SR₂₇, S(O)R₂₇, or S(O)₂R₂₇,
   (h) mono-substituted or di-substituted phenyl, wherein the substituents are selected from:
      (i) —CF₃,
      (ii) —CN,
      (iii) F, Cl, Br, or I,
      (iv) C₁₋₆ alkyl and
      (v) SR₂₇, S(O)R₂₇, or S(O)₂R₂₇,
or R₁₉ and R₂₀ together form an oxo group,
or R₂₁ and R₂₂ together form an oxo group,
or R₂₃ and R₂₄ together form an oxo group,
or R₁₉ and R₂₀ are joined so that together with the carbon atom to which they are attached there is formed a saturated 3, 4, 5, 6 or 7 membered hydrocarbon ring;
or R₂₀ and R₂₁ are joined so that together with the carbon atom to which they are attached there is formed a saturated 3, 4, 5, 6 or 7 membered hydrocarbon ring;
or R₂₀ and R₂₃ are joined so that together with the carbon atom to which they are attached there is formed a saturated 3, 4, 5, 6 or 7 membered hydrocarbon ring;
or R₂₁ and R₂₂ are joined so that together with the carbon atom to which they are attached there is formed a saturated 3, 4, 5, 6 or 7 membered hydrocarbon ring;
or R₂₁ and R₂₃ are joined so that together with the carbon atom to which they are attached there is formed a saturated 3, 4, 5, 6 or 7 membered hydrocarbon ring;
or R₂₄ and R₂₅ are joined so that together with the carbon atom to which they are attached there is formed a saturated 3, 4, 5, 6 or 7 membered hydrocarbon ring;
R₂₆ is O or S;

R₂₇ is methyl, ethyl, mono-substituted benzyl or di-substituted benzyl, wherein the substituents are selected from:
(a) CF₃,
(b) CN,
(c) F, Cl, Br, I, and
(d) C₁₋₆ alkyl;
R₂₈ and R₂₉ are independently selected from:
   (a) H,
   (b) C₁₋₃ alkyl
   (c) C₃₋₆ cycloalkyl
   (d) —OR₃₂,
   (e) —C(O)R₃₀
   (f) —S(O)₂R₃₁,
   (g) a mono-substituted C₂₋₄ alkyl, wherein the substituent is selected from:
      (i) hydroxy,
      (ii) amino,
      (iii) methylamino,
      (iv) dimethyl amino
   (h) an unsubstituted, mono-substituted, or disubstituted phenyl, benzyl or pyridyl group, wherein the substituents are selected from:
      (i) H,
      (ii) CF₃,
      (iii) CN,
      (iv) F, Cl, Br, I, and
      (v) C₁₋₆ alkyl,
or R₂₈ and R₂₉ are joined so that together with the nitrogen atom to which they are attached there is formed a 3, 4, 5, 6, or 7-membered rig, optionally including one or two additional heteroatoms, wherein the additional heteroatoms are selected from N, O, and S, wherein a carbon atom may optionally be substituted with an oxo group and a sulfur atom of the ring may optionally substituted with two oxo groups;
R₃₀ is selected from:
   (a) H,
   (b) C₁₋₄ alkyl,
   (c) CF₃,
   (d) an unsubstituted, mono-substituted, or disubstituted phenyl or benzyl group, wherein the substituents are selected from:
      (i) CF3,
      (ii) CN,
      (iii) F, Cl, Br, I, and
      (iv) C₁₋₆ alkyl;
R₃₁ is selected from:
   (a) C₁₋₄ alkyl,
   (b) CF₃, and
   (c) an unsubstituted, mono-substituted, or disubstituted phenyl or benzyl group, wherein the substituents are selected from:
      (i) CF₃,
      (ii) CN,
      (iii) F, Cl, Br, I, and
      (iv) C₁₋₆ alkyl.

In various embodiments, the inventions features: a compound having Formula I wherein R₂ is A and R_{3A} and R_{3B} together form an oxo group; a compound having Formula I wherein R₂ is A and both R_{3A} and R_{3B} are both H; a compound having Formula I wherein R₇ is H or methyl; a compound having Formula I wherein R_{4B} and/or or R_{4C} is a halogen (e.g., Br); a compound having Formula I wherein R₂ is A and R_{1A} is hydroxy.

The invention also features a compound having Formula I wherein R₂ is B and a compound having Formula I wherein R₂ is C. In certain embodiments, where R₂ is B or C, R_{1A} is hydroxy.

The invention also features a compound having Formula I wherein R_{1A} is selected from: a prodrug of a hydroxy moiety, a hydroxy group, —OC(O)CH₃, an ester having a C₁, branched or straight chain alkyl group, phosphate ester having C₁₋₆ branched or straight alkyl groups, a carbamate having C₁₋₆ branched or straight alkyl groups, and a carbonate group having a C₁₋₆ branched or straight chain alkyl group; R₂ is A wherein R₆ and R₇ are H; R_{3A} and R_{3B} together form an oxo group or are both H; at least one of R_{1B} and R_{1C} is a halogen; at least one of R_{4A}, R_{4B}, R_{4C}, R_{4D}, and R_{4E} is a halogen or one or both of R_{4B} and R_{4C} are halogen and R_{4A}, R_{4D}, and R_{4E} are H or one or both of R_{1B} and R_{1C} is a halogen and R_{1D} is hydrogen.

The invention also features compositions comprising a compound having Formula I, wherein the composition contains no more than 0.0001%, 0.001%, 0.01%, 0.1%, 0.3%, 0.5%, 0.9%. 1.9%, 5.0%, or 10% by weight other compounds.

The invention also features a pharmaceutical composition comprising: (a) a non-toxic therapeutically effective amount of any of the forgoing compounds having Formula I, and (b) a pharmaceutically acceptable carrier. In a preferred embodiment, the composition does not comprise indomethacin. In other preferred embodiments of the pharmaceutical composition, R₂ is A and/or R_{1A} is hydroxy.

In other embodiments, the invention features: a pharmaceutical composition comprising a pharmaceutically acceptable carrier and a non-toxic therapeutically effective amount of a compound having Formula I, e.g., a compound selected from those depicted above, for example: (a) [1-(4-chlorobenzoyl)-5-hydroxy-2-methyl-1H-indol-3-yl]acetic acid, (b) (2R)-2-[1-(4-chlorobenzoyl)-5-hydroxy-2-methyl-1H-indol-3-yl]propanoic acid; (c) (2S)-2-[1-(4-chlorobenzoyl)-5-hydroxy-2-methyl-1H-indol-3-yl]propanoic acid; and (d) 2-[1-(4 chlorobenzoyl)-5-hydroxy-2-methyl-1H-indol-3-yl]propanoic acid. In certain embodiments the composition does not comprise indomethacin. In other embodiments the composition comprises: 2-[1-(4-chlorobenzoyl)-5-hydroxy-2-methyl-1H-indol-3-yl]propionic acid wherein at least at least 75% of the 2-[1-(4-chlorobenzoyl)-5-hydroxy-2-methyl-1H-indol-3-yl]propionic acid is (2R)-2-[1-(4-chlorobenzoyl)-5-hydroxy-2-methyl-1H-indol-3-yl]propionic acid and no more than 25% is (2S)-2-[1-(4-chlorobenzoyl)-5-hydroxy-2-methyl-1H-indol-3-yl]propionic acid. In various preferred embodiments, at least at least 85% of the 2-[1-(4-chlorobenzoyl)-5-hydroxy-2-methyl-1H-indol-3-yl]propionic acid is (2R)-2-[1-(4-chlorobenzoyl)-5-hydroxy-2-methyl-1H-indol-3-yl]propionic acid; and at least at least 95% of the 2-[1-(4-chlorobenzoyl)-5-hydroxy-2-methyl-1H-indol-3-yl]propionic acid is (2R)-2-[1-(4-chlorobenzoyl)-5-hydroxy-2-methyl-1H-indol-3-yl]propionic acid.

The invention also features a method of treating a disorder associated with unwanted COX-2 activity, the method comprising providing a patient with a therapeutically effective serum concentration of a compound having Formula I in the absence of measurable serum indomethacin. In various embodiments of the method: the disorder is an inflammatory disorder, R₂ is A and R₃ and R_{3′} are H or together form an oxo group; R₂ is A and R₃ and R_{3′}, are H; R₇ is H or methyl; R₄ is a halogen; R₂ is B and R₁ is hydroxy or a group that is metabolized to hydroxy; R₂ is C and R₁ is hydroxy or a group that is metabolized to hydroxy.

The invention also features treating a patient, e.g., a patient suffering from inflammation or pain or both inflammation and pain by administering a pharmaceutical composition comprising a compound having Formula I and a pharmaceutically acceptable carrier. The invention further features a method for treating a patient suffering from enuresis by administering a composition comprising compound I. In certain embodiments of the method the patient is not treated a the same time with indomethacin. In certain embodiments the pain is nociceptive, neuropathic or inflammatory in origin or some combination thereof.

In other embodiments, the method comprises administering to the patient a compound of the invention and an agent for the treatment of inflammation, pain, enuresis or fever, e.g., a NSAID other than indomethacin.

The invention includes prodrugs of compounds having Formula I (other than indomethacin) that are converted in vivo so that a hydroxyl group is present at R_{1A}. Thus, the invention also features a compound having Formula I wherein the prodrug of a hydroxy moiety (e.g., variations of compound in which R_{1A} is a hydroxy) is selected from: (a) an ester having a C₁₋₆ branched or straight chain alkyl group, (b) phosphate ester having C₁₋₆ branched or straight chain alkyl groups, (c) a carbamate having C₁₋₆ branched or straight chain alkyl groups, (d) a carbonate group having a C₁₋₆ branched or straight chain alkyl group.

Thus, R_{1A} can be, for example, 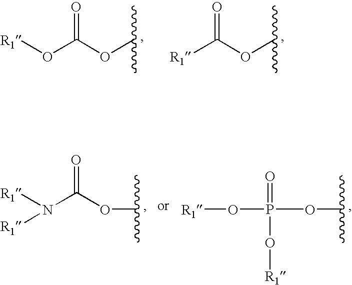
wherein R_{1″} is H or a C₁₋₆ straight chain or branched alkyl, alkenyl, alkynyl, aryl, cycloalkyl, or arylalkyl that is optionally singly or multiply substituted, e.g., a C₁-C₆ haloalkyl. Particularly useful are compound in which R_{1″} is selected from: H and a substituted or unsubstituted C₁ alkyl, a C₂ alkyl, a C₃ alkyl or a C₄ alkyl.

The invention features compounds having Formula II or Formula IIa or a pharmaceutically acceptable salt thereof, pharmaceutical compositions comprising such compounds and methods for treating a patient by administering such compounds pharmaceutical compositions alone or in combination with one or more other therapeutic agents.

Thus, the invention also features compound having Formula II 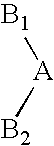
wherein:
A is a 5-membered or 6-membered substituted or unsubstituted heteroaryl (e.g., monocyclic) or heterocyclyl (e.g., monocyclic) ring having one or two heteroatoms selected from the group consisting of O, S, and N and having from 1 or 2 independent substituents when the ring is 5-membered and substituted with 1 to 4 independent substituents when the ring is 6-membered and substituted; and
each of B₁ and B₂ is the same or different substituted or unsubstituted 6-membered aryl or heteroaryl ring;
and salts thereof.

In certain embodiments, A has two heteroatoms and the heteroatoms are N and O or N and S.

In various embodiments, each of B₁ and B₂ is independently a phenyl or pyridyl group; at least one of B₁ and B₂ is singly or independently multiply substituted and the substituents are selected from: a methyl group optionally, independently substituted with one or more halogen, an ethyl group optionally, independently substituted with one or more halogen, a halogen, —OH, —OCH₃, optionally, independently substituted with one or more halogen, and —SCH₃ optionally, independently substituted with one or more halogen; at least one of B₁ and B₂ is singly or independently multiply substituted and the substituents are selected from: a methyl group, an ethyl group, a halogen, —OCF₃, —OCF₂H, —SCF₃, —OH, —OCH₃, and —SCH₃; A is selected from the group consisting of oxazolyl, isoxazolyl, thiazolyl, isothiazolyl, pyrazolidinyl, pyrazolyl, furanyl, and pyridinyl; and each of B₁ and B₂ is a singly or multiply substituted-phenyl group; and each of B₁ and B₂ is independently singly or multiply substituted and the substituents are selected from hydroxyl and halogen.

In certain embodiments one or both of B₁ and B₂ is:

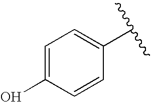

In certain embodiments, the compound having Formula II selected from the group consisting of: 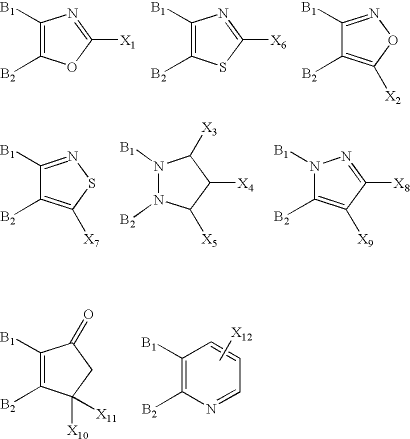
wherein
each X₁-X₁₂ is independently: H, halogen, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₆ alkoxy, oxo, substituted or unsubstituted C₂₋₁₂ alkenyloxy, substituted or unsubstituted C₅₋₁₀ cycloalkenyloxy, substituted or unsubstituted (C₂₋₁₂ alkynyl)oxy, (C₁₋₆ alkyl)oxy(C₁₋₆ alkyl), substituted or unsubstituted C₆₋₁₂ aryloxy, (C₃₋₆ heteroaryl)-(C₁₋₆ alkyl)oxy, (C₁₋₁₂ alkyl)thio, substituted or unsubstituted (C₁₋₄ alkyl)-thio-(C₁₋₄ alkyl), substituted or unsubstituted C₆-C₁₀ aryl, substituted or unsubstituted styryl, substituted or unsubstituted C₃₋₁₂ heteroaryl, substituted or unsubstituted C₄₋₈ heterocyclic, wherein the substituents are selected from the group consisting of hydroxy, halo, C₁₋₄ alkyl, C₁₋₄ trihaloalkyl, C₁₋₆ alkoxy, C₁₋₄ trihaloalkoxy, bivalent oxy(C₁₋₆)alkyloxy, (C₁₋₆) acylamino, (C₁₋₆) acylthio, amino, and azido; or R⁵ and R⁶ form a C₅-C₁₀ heteroaryl ring, and each of R⁴, R⁷, and R⁸ is, independently, hydroxy, halo, C₁₋₄ alkyl, C₁₋₄ trihaloalkyl, C₁₋₆ alkoxy, or C₁₋₄ trihaloalkoxy.

In various embodiments: one of B₁ and B₂ is substituted and the other is unsubstituted; both B₁ and B₂ are substituted; both B₁ and B₂ are unsubstituted; one of B₁ and B₂ is singly substituted and the other is unsubstituted; and one or both of B₁ and B₂ are independently substituted and the substituents are selected from: 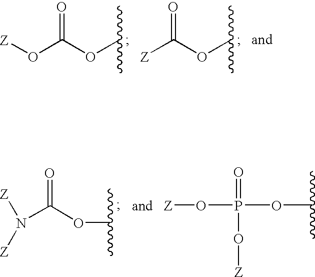
wherein each Z is independently H or a C₁₋₆ straight chain or branched alkyl, alkenyl, alkynyl, aryl, cycloalkyl, or arylalkyl that is optionally singly or multiply substituted.

In certain embodiments, Z is independently H or a C₁₋₆ straight chain or branched alkyl, alkenyl, alkynyl, aryl, cycloalkyl, or arylalkyl that is optionally singly or multiply substituted with a halogen; and Z is independently selected from: H and a substituted or unsubstituted C₁ alkyl, C₂ alkyl, C₃ alkyl or C₄ alkyl.

The invention also features a compound having Formula IIa: 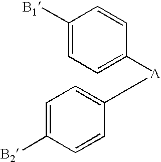
wherein
A is a 5-membered or 6-membered substituted or unsubstituted heteroaryl or heterocyclyl ring having one or two heteroatoms selected from the group consisting of O, S, and N and having from 1 or 2 independent substituents when the ring is 5-membered and substituted and 1 to 4 independent substituents when the ring is 6-membered and substituted; and one or both of B_{1′} and B_{2′} are selected from H, —OH, 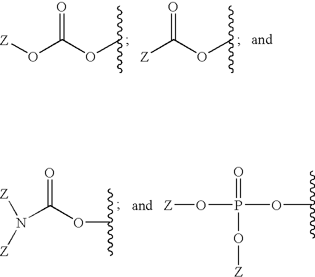
wherein each Z is independently H or a C₁₋₆ straight chain or branched alkyl, alkenyl, alkynyl, aryl, cycloalkyl, or arylalkyl that is optionally singly or multiply substituted; and
and salts thereof.

In various embodiments, A is selected from: 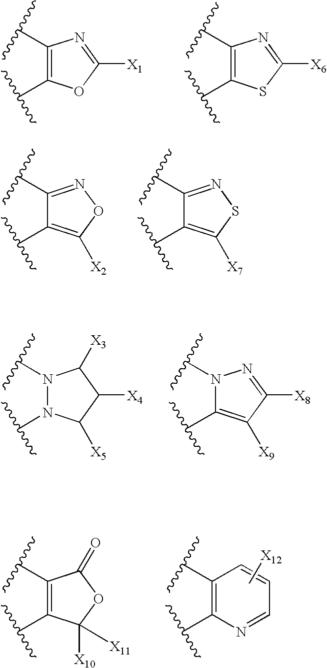
wherein each X₁-X₁₂ is independently: H, halogen, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₆ alkoxy, oxo, substituted or unsubstituted C₂₋₁₂ alkenyloxy, substituted or unsubstituted C₅₋₁₀ cycloalkenyloxy, substituted or unsubstituted (C₂₋₁₂ alkynyl)oxy, (C₁₋₆ alkyl)oxy(C₁₋₆ alkyl), substituted or unsubstituted C₆₋₁₂ aryloxy, (C₃₋₆ heteroaryl)-(C₁₋₆ alkyloxy, (C₁₋₁₂ alkyl)thio, substituted or unsubstituted (C₁₋₄ alkyl)-thio-(C₁₋₄ alkyl), substituted or unsubstituted C₆-C₁₀ aryl, substituted or unsubstituted styryl, substituted or unsubstituted C₃₋₁₂ heteroaryl, substituted or unsubstituted C₄₋₈ heterocyclic, wherein the substituents are selected from the group consisting of hydroxy, halo, C₁₋₄ alkyl, C₁₋₄ trihaloalkyl, C₁₋₆ alkoxy, C₁₋₄ trihaloalkoxy, bivalent oxy(C₁₋₆)alkyloxy, (C₁₋₆) acylamino, (C₁₋₆) acylthio, amino, and azido; or R⁵ and R⁶ form a C₅-C₁₀ heteroaryl ring, and each of R⁴, R⁷, and R⁸ is, independently, hydroxy, halo, C₁₋₄ alkyl, C₁₋₄ trihaloalkyl, C₁₋₆ alkoxy, or C₁₋₄ trihaloalkoxy.

In other embodiments, A is selected from:

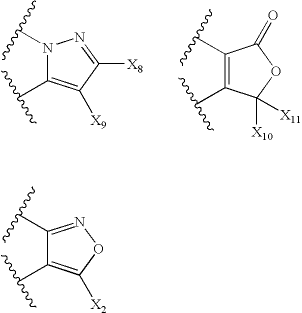

In certain embodiments, X₂ is selected from H, CH₃, COOH, —CH₂—CH₂—COOH, —CH₂—COOH and CF₃; X₁₀ and X₁₁ are H or one or both of X₁₀ and X₁₁ are CH₃ or CF₃; X₉ is missing; and X₈ is CH₃ or CF₃; and each X₁-X₁₂ is independently: H, halogen, substituted or unsubstituted C₁-C₃ alkyl, substituted or unsubstituted C₁-C₃ alkoxy, or oxo.

The invention further features the following compounds: selected from: 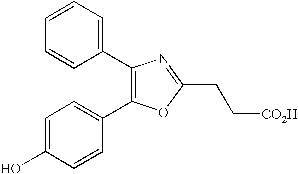
3-[5-(4-hydroxyphenyl)-4-phenyl-1,3-oxazol-2-yl]propionic acid 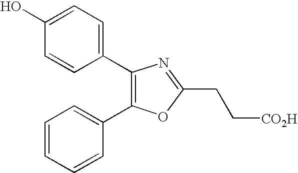
3-[4-(4-hydroxyphenyl)-5-phenyl-1,3-oxazol-2-yl]propionic acid 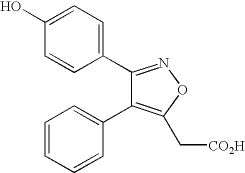
[3-(4-hydroxyphenyl)-4-phenylisoxazol-5-yl]acetic acid 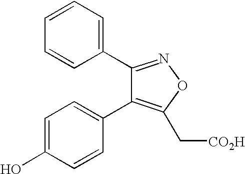
[4-(4-hydroxyphenyl)-3-phenylisoxazol-5-yl]acetic acid 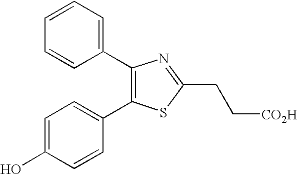
3-[5-(4-hydroxyphenyl)-4-phenyl-1,3-thiazol-2-yl]propionic acid 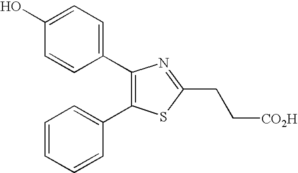
3-[4-(4-hydroxyphenyl)-5-phenyl-1,3-thiazol-2-yl]propionic acid 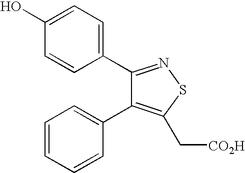
[3-(4-hydroxyphenyl)-4-phenylisothiazol-5-yl]acetic acid 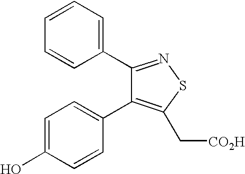
[4-(4-hydroxyphenyl)-3-phenylisothiazol-5-yl]acetic acid 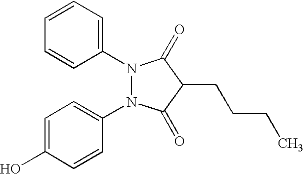
4-butyl-1-(4-hydroxyphenyl)-2-phenylpyrazolidine-3,5-dione 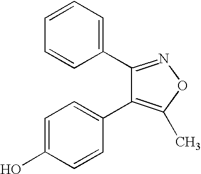
4-(5-methyl-3-phenylisoxazol-4-yl)phenol 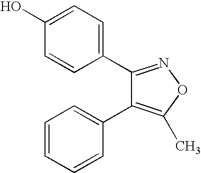
4-(5-methyl-4-phenylisoxazol-3-yl)phenol 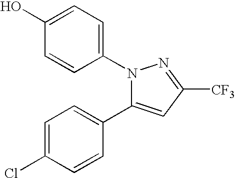
-1-(4-hydroxyphenyl)-3-(trifluoromethyl)-5-(4-chlorophenyl)-pyrazole 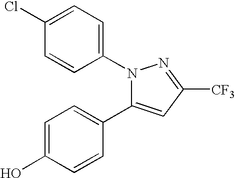
1-(4-chloroyphenyl)-3-(trifluoromethyl)-5-(4-hydroxyphenyl)-pyrazole 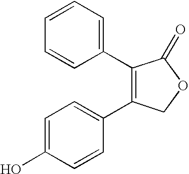
4-(4-hydroxyphenyl)-3-phenylfuran-2(5H)-one 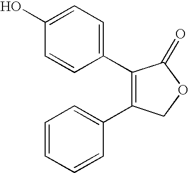
3-(4-hydroxyphenyl)-4-phenylfuran-2(5H)-one 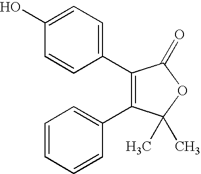
3-(4-hydroxyphenyl)-5,5-dimethyl-4-phenylfuran-2(5H)-one 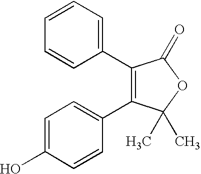
4-(4-hydroxyphenyl)-5,5-dimethyl-3-phenylfuran-2(5H)-one 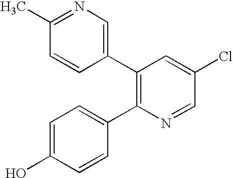
4-(5-chloro-6′-methyl-3,3′-bipyridin-2-yl)phenol 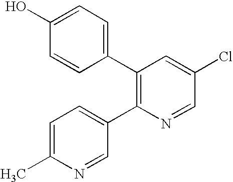
4-(5-chloro-6′-methyl-2,3′-bipyridin-3-yl)phenol 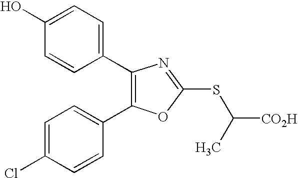
2-{[5-(4-chlorophenyl)-4-(4-hydroxyphenyl)-1,3-oxazol-2-yl]thio}propionic acid 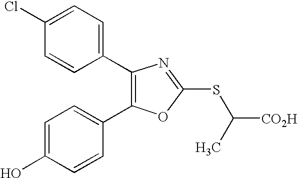
2-{[4-(4-chlorophenyl)-5-(4-hydroxyphenyl)-1,3-oxazol-2-yl]thio}propionic acid 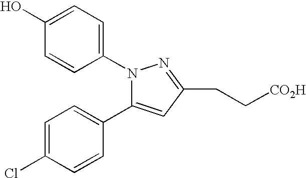
3-[5-(4-chlorophenyl)-1-(4-hydroxyphenyl)-1H-pyrazol-3-yl]propionic acid 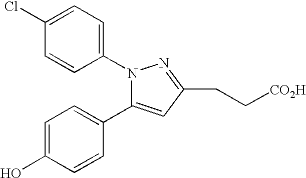
3-[1-(4-chlorophenyl)-5-(4-hydroxyphenyl)-1H-pyrazol-3-yl]propionic acid.

The invention also features a pharmaceutical composition comprising the compound of Formula II or Formula IIa and a pharmaceutically acceptable carrier. In some embodiments, the pharmaceutical composition further comprises a second compound having anti-inflammatory activity and/or antinociceptive activity. In certain embodiments the second compound is a COX-2 inhibitor (e.g., a selective COX-2 inhibitor).

The invention features a method for treating a disorder associated with unwanted COX-2 activity, the method comprising providing a patient with a therapeutically effective serum concentration of a compound of Formula II or Formula IIa.

The invention features a method for treating a disorder associated with unwanted COX-2 activity, the method comprising administering the pharmaceutical composition comprising a compound of Formula II or Formula IIa.

The invention features a method for treating inflammation, the method comprising providing a patient with a therapeutically effective serum concentration of a compound of Formula II or Formula IIa.

The invention features a method for treating inflammation, the method comprising administering the pharmaceutical composition comprising a compound of Formula II or Formula IIa.

The invention features a method for treating pain (e.g., nociceptive pain or neuropathic pain), the method comprising providing a patient with a therapeutically effective serum concentration of a compound of Formula II or Formula IIa.

The invention features a method for treating pain (e.g., nociceptive pain or neuropathic pain), the method comprising administering the pharmaceutical composition comprising a compound of Formula II or Formula IIa.

The invention features a method for treating anxiety, the method comprising providing a patient with a therapeutically effective serum concentration of a compound of Formula II or Formula IIa.

The invention features a method for treating anxiety, the method comprising administering the pharmaceutical composition comprising a compound of Formula II or Formula IIa.

The invention features a method for treating a sleep disorder (e.g., insomnia), the method comprising providing a patient with a therapeutically effective serum concentration of a compound of Formula II or Formula IIa.

The invention features a method for treating a sleep disorder (e.g., insomnia), the method comprising administering the pharmaceutical composition comprising a compound of Formula II or Formula IIa.

The invention includes a compound of Formula II or Formula IIa, wherein the compound exhibits an IC₅₀ for FAAH that is less than 50 μM or less than 10 μM.

Where B is a substituted phenyl, in some embodiments the substituants are preferably selected from hydroxy, halogen (Br, Cl, and Fl or Br, Cl, Fl, and I), methyl, and ethyl.

The invention includes salts, particularly physiologically acceptable salts, and solvates of the compounds having Formula I or Formula II or Formula IIa. Solvates are forms of the compounds in which the compound forms a complex with solvent molecules by coordination in the solid or liquid states. Hydrates are a special form of solvate in which the compound is coordinated with water.

Certain compound within Formula I or Formula II or Formula IIa may exist in stereoisomeric forms such as enantiomers, diastereomers and mixtures thereof. Mixtures can be separated into stereoisomerically pure constituents. Certain compounds may be tautomeric, and the invention encompasses the various tautomeric mixtures.

The invention also features compositions comprising a compound having Formula II or Formula IIa, wherein the composition contains no more than 0.0001%, 0.001%, 0.01%, 0.1%, 0.3%, 0.5%, 0.9%. 1.9%, 5.0%, or 10% by weight other compounds.

The invention also features a pharmaceutical composition comprising: (a) a non-toxic therapeutically effective amount of having Formula II or Formula IIa, e.g., one of the compounds depicted above and (b) a pharmaceutically acceptable carrier. In certain embodiments, the composition includes a second active compound, the second active compound having anti-inflammatory activity and/or anti-nociceptive activity, e.g., the second active compound is a COX-2 inhibitor or an analgesic.

The invention also features a method of treating a disorder associated with unwanted COX-2 activity, the method comprising providing a patient with a therapeutically effective serum concentration of a compound having Formula II or Formula IIa which is an inhibitor of COX-2.

The invention also features treating a patient, e.g., a patient suffering from unwanted inflammation or pain or both unwanted inflammation and pain by administering a pharmaceutical composition comprising a compound having Formula II or Formula IIa and a pharmaceutically acceptable carrier. In certain embodiments the pain is nociceptive, neuropathic or inflammatory in origin or some combination thereof. The invention further features a method for treating a patient suffering from enuresis by administering a composition comprising a compound having Formula II or Formula IIa.

In other embodiments, the method comprises administering to the patient a compound of the invention and a second active compound for the treatment of inflammation, pain or fever, e.g., COX-2 inhibitor such as a NSAID.

The invention includes prodrugs of compounds having Formula II or Formula IIa.

The invention features methods for treating a subject with a pharmaceutical composition comprising a compound of Formula I or Formula II or Formula IIa and a pharmaceutically acceptable carrier. The subject can be a mammal, preferably a human. Identifying a subject in need of such treatment can be in the judgment of a subject or a health care professional and can be subjective (e.g., opinion) or objective (e.g., measurable by a test or diagnostic method).

The compounds of the invention have anti-inflammatory activity, analgesic activity or both. The anti-inflammatory activity arises from the ability to inhibit COX-2. Certain of the compounds inhibit COX-2 to a greater extent than COX-1 in vivo. Some of the compounds of Formula I and Formula II and Formula IIa have analgesic activity. The analgesic activity does not necessarily arise from the inhibition of COX-2 activity. Instead the analgesic activity may be due to a mechanism other than COX-2 inhibition. Thus, analgesic activity may arise from one or more of: inhibiting COX-2 activity, increasing the level of 3α,5α-THP, inhibiting fatty acid amidohydrolase, or activating CB₁. Thus, the invention features a compound having Formula I or II and having the ability to: inhibit COX-2 activity, increase the level of 3α,5α-THP, inhibit fatty acid amidohydrolase, or activate CB₁

The compounds of the invention are expected to have a number of advantages when compared to indomethacin, which is considered a highly effective COX-2 inhibitor among the NSAIDs. First, because the compounds of the invention are relatively selective for inhibition of COX-2 over COX-1, they are in general expected to elicit few side-effects when used for treatment of inflammation or pain when compared to indomethacin or a COX inhibitor that is less selective for COX-2. Thus, they are expected to cause less irritation to the gastrointestinal tract.

Many of the compounds of the invention are expected to exhibit better solubility than other COX-2 inhibitors and this will allow more rapid onset of anti-inflammatory and analgesic activity. In addition, many of the compounds of the invention will exhibit lower binding to plasma proteins resulting in a higher and more consistent effective level of activity.

Many of the compounds of the invention are expected to be suitable for parenteral administration because they are relatively soluble in aqueous solutions. This is particular desirable where oral administration is difficult or impossible, e.g., during surgery or in a post-surgical setting.

The term “treating” or “treated” refers to administering a compound described herein to a subject with the purpose to cure, heal, alleviate, relieve, alter, remedy, ameliorate, improve, or affect a disease, the symptoms of the disease or the predisposition toward the disease.

“An effective amount” refers to an amount of a compound that confers a therapeutic effect on the treated subject. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, alternatively from about 1 to about 50 mg/Kg. Effective doses will also vary depending on route of administration, as well as the possibility of co-usage with other agents.

The term “mammal” includes, for example, mice, hamsters, rats, cows, sheep, pigs, goats, horses, monkeys, dogs (e.g., Canis familiaris), cats, rabbits, guinea pigs, and primates, including humans.

The term “prodrug” refers to compounds which are drug precursors which, following administration and absorption, release the drug in vivo through a metabolic process. Exemplary prodrugs include acyl amides of the amino compounds of this invention such as amides of alkanoic (C₁₋₆)acids, amides of aryl acids (e.g., benzoic acid) and alkane (C₁₋₆)dioic acids.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl). The terms “arylalkyl” or “aralkyl” refer to an alkyl moiety in which an alkyl hydrogen atom is replaced by an aryl group. Examples of “arylalkyl” or “aralkyl” include benzyl and 9-fluorenyl groups.

The terms “alkylamino” and “dialkylamino” refer to —NH(alkyl) and —N(alkyl)₂ radicals respectively. The term “aralkylamino” refers to a —NH(aralkyl) radical. The term “alkoxy” refers to an —O-alkyl radical. The term “mercapto” refers to an SH radical. The term “thioalkoxy” refers to an —S-alkyl radical.

The term “aryl” refers to an aromatic monocyclic, bicyclic, or tricyclic hydrocarbon ring system, wherein any ring atom capable of substitution can be substituted by a substituent. Examples of aryl moieties include, but are not limited to, phenyl, naphthyl, and anthracenyl.

The term “cycloalkyl” as employed herein includes saturated cyclic, bicyclic, tricyclic, or polycyclic hydrocarbon groups having 3 to 12 carbons, wherein any ring atom capable of substitution can be substituted by a substituent. Examples of cycloalkyl moieties include, but are not limited to, cyclopentyl, norbornyl, and adamantyl.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “oxo” refers to an oxygen atom, which forms a carbonyl when attached to carbon, an N-oxide when attached to nitrogen, and a sulfoxide or sulfone when attached to sulfur.

The term “alkoxy” refers to an —O-alkyl radical. Similarly, the alkyl portion of alkoxy substituents may be cyclic, acyclic, or combinations thereof, or branched or unbranched. The cyclic alkyl portion may have one or more rings, which may be bridged or fused.

The term “alkenyl” refers to a straight or branched hydrocarbon chain containing 2-12 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. The term “alkynyl” refers to a straight or branched hydrocarbon chain containing 2-12 carbon atoms and having one or more triple bonds. Some examples of a typical alkynyl are ethynyl, 3-hexynyl, and propargyl. One of the triple bond carbons may optionally be the point of attachment of the alkynyl substituent.

The term “heterocyclyl” or “heterocyclic” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The heteroatom may optionally be the point of attachment of the heterocyclyl substituent. Any ring atom can be substituted. The heterocyclyl groups can contain fused rings. Fused rings are rings that share a common carbon atom. Examples of heterocyclyl include, but are not limited to, tetrahydrofuranyl, tetrahydropyranyl, piperidinyl, morpholino, pyrrolinyl and pyrrolidinyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Any ring atom can be substituted.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Suitable substituents include, without limitation, alkyl, alkenyl, alkynyl, alkoxy, acyloxy, halo, hydroxy, cyano, nitro, amino, SO₃H, sulfate, phosphate, perfluoroalkyl, perfluoroalkoxy, methylenedioxy, ethylenedioxy, carboxyl, oxo, thioxo, imino (alkyl, aryl, aralkyl), S(O)ₙalkyl where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents.

The details of one or more embodiments of the invention are set forth in the accompanying drawings and the description below. Other features, objects, and advantages of the invention will be apparent from the description and drawings, and from the claims. The patents, patent applications, and publications referenced herein are hereby incorporated by reference in their entirety.

### DESCRIPTION OF DRAWINGS

FIG. 1 is a graph depicting the results of assays measuring the influence of indomethacin on COX-1 activity and COX-2 activity.

FIG. 2 is a graph depicting the results of assays measuring the influence of desmethylindomethacin on COX-1 activity and COX-2 activity.

### DETAILED DESCRIPTION

The invention features compounds having Formula I, e.g., desmethylindomethacin (indole-3-acetic acid, 1-(p-chlorobenzoyl)-5-hydroxy-2-methyl-(7CI, 8CI); 1-(p-Chlorobenzoyl)-5-hydroxy-2-methylindole-3-acetic acid; 5-Hydroxyindomethacin; Demethylindomethacin; O-desmethylindomethacin; 1H-Indole-3-acetic acid, 1-(4-chlorobenzoyl)-5-hydroxy-2-methyl; CAS Registry 2504-32-7) and derivatives thereof that are selective inhibitors of COX-2 as well as compounds that are metabolized to desmethylindomethacin or a derivative thereof that is a selective inhibitor of COX-2.

The invention features compounds of Formula I, Formula II, or Formula IIa or related prodrugs thereof that inhibit COX-2 and/or FAAH. The COX-2 inhibitors are selective COX-2 inhibitors in that they are selective for inhibition of COX-2 as compared to COX-1. Certain of the FAAH inhibitors are selective for inhibition of FAAH relative to both COX-2 and COX-1. Certain of the COX-2 inhibitors, in addition to being selective for COX-2 relative to COX-1, are selective for COX-2 relative to FAAH.

Useful selective COX-2 inhibitors are those which inhibit COX-2 activity at physiological concentrations where COX-1 activity is not significantly inhibited. Thus, selective COX-2 inhibitors can have an IC₅₀ for COX-1 that is at least 2-, 5-, 10-, 15-, 20-, 100-, 500-, 1,000-fold greater than the IC₅₀ for COX-2. Particularly desirable are compounds that do not significantly inhibit COX-1 at a therapeutically effective concentration, e.g., a concentration effective to reduce pain or inflammation attributable to COX-2 associated prostaglandin production. Useful compounds include those having an IC₅₀ for COX-2 of less than about 2.0, 1.5, 1.0, 0.5, 0.4, 0.3, 0.2, 0.1, 0.08, 0.06, 0.04, 0.02, or 0.01 μM, and have an IC₅₀ for COX-1 of greater than about 1, 5, 10, 15, 20, 40 or 100 μM. In certain embodiments the COX-2 IC₅₀ for a compound is less than 20, 10, 5, 3, 2, 1, 0.5, 0.4, 0.3, 0.2, 0.1 or 0.05 times the COX-2 IC₅₀ for indomethacin in the same assay. In certain embodiments the COX-1 IC₅₀ for a compound is at least 2, 5, 10, 25, 50, 100, 500, 1000 or more times the COX-1 C₅₀ for indomethacin in the same assay. In certain embodiments, the selectivity for COX-2 over COX-1 for a compound is greater than 3, 5, 10, 50, 100, 200, 500 or 1000 times the selectivity of indomethacin in the same assays.

Certain useful selective FAAH inhibitors include those which inhibit FAAH activity at a physiological concentration at which both COX-1 and COX-2 activity are not significantly inhibited. Thus, certain useful compounds have an IC₅₀ for COX-1 and COX-2 that is at least 2-, 5-, 10-, 15-, 20-, 100-, 500-, 1,000-fold greater than the IC₅₀ for FAAH. Certain FAAH inhibitors do not measurably inhibit COX-1 and COX-2 at a therapeutically effective concentration, e.g., a concentration effective to reduce pain. Useful FAAH inhibitory compounds include those having an IC₅₀ for FAAH of less than about 80, 60, 40, 20, 10, 5, 2.0, 1.5, 1.0, 0.5, 0.4, 0.3, 0.2, 0.1, 0.08, 0.06, 0.04, 0.02, or 0.01 μM, and have an IC₅₀ for COX-1 and COX-2 of greater than about 1, 5, 10, 15, 20, 50, 100, 200, or 400 μM. In certain embodiments, the IC₅₀ for FAAH for a compound is no more than about 5, 1, 0.1, 0.05, 0.01 or 0.001 times the IC₅₀ for FAAH of indomethacin in the same assay.

Of course, other useful FAAH inhibitors also inhibit COX-2 at physiological concentrations at which COX-1 activity is not significantly inhibited. Particularly desirable are compounds that do not measurably inhibit COX-1 at a therapeutically effective concentration, e.g., a concentration effective to reduce pain. Useful compounds can include those having an IC₅₀ for FAAH of less than about 80, 60, 40, 20, 10, 5, 2.0, 1.5, 1.0, 0.5, 0.4, 0.3, 0.2, 0.1, 0.08, 0.06, 0.04, 0.02, or 0.01 μM, an IC₅₀ for COX-2 of less than about 2.0, 1.5, 1.0, 0.5, 0.4, 0.3, 0.2, 0.1, 0.08, 0.06, 0.04, 0.02, or 0.01 μM, and an IC₅₀ for COX-1 of greater than about 1, 5, 10, 15, or 20 μM. In certain embodiments, the COX-2 IC₅₀ for such a FAAH inhibitor is less than 20, 10, 5, 3, 2, 1, 0.5, 0.4, 0.3, 0.2, 0.1 or 0.05 times the COX-2 IC₅₀ for indomethacin in the same assay. In certain embodiments, the COX-1 IC₅₀ for such a FAAH inhibitor is at least 2, 5, 10, 25, 50, 100, 500, 1000 or more times the COX-1 IC₅₀ for indomethacin in the same assay.

Certain useful selective COX-2 inhibitors include those which inhibit COX-2 activity at physiological concentrations where FAAH activity is not significantly inhibited. Thus, certain useful compounds do not significantly inhibit FAAH at a therapeutically effective concentration, e.g., a concentration effective to reduce pain. Certain useful compounds have an IC₅₀ for COX-2 of less than about 2.0, 1.5, 1.0, 0.5, 0.4, 0.3, 0.2, 0.1, 0.08, 0.06, 0.04, 0.02, or 0.01 μM, and have an IC₅₀ for FAAH of greater than about 5, 10, 15, 20, 50, 100, 200 or 400 μM. Of course, other useful COX-2 inhibitors also inhibit FAAH at therapeutically relevant doses, i.e., they are not particularly selective for COX-2 over FAAH. In certain embodiments the COX-2 IC₅₀ for a compound is less than 20, 10, 5, 3, 2, 1, 0.5, 0.4, 0.3, 0.2, 0.1 or 0.05 times the COX-2 IC₅₀ for indomethacin in the same assay. In certain embodiments the COX-1 IC₅₀ for a compound is at least 2, 5, 10, 25, 50, 100, 500, 1000 or more times the COX-1 IC₅₀ for indomethacin in the same assay.

### EXAMPLE 1

### COX-1 and COX-2 Purified Enzyme Assays

The COX-1 and COX-2 inhibitory activities of indomethacin, desmethylindomethacin, and desbenzoylindomethacin were compared using a test kit available from Cayman Chemical (Ann Arbor, Mich.). Because COX-1 and COX-2 convert arachidonic acid to prostaglandin H₂ (PGH₂), one can assess COX inhibitory activity of a test compound by measuring the effect of the compound on PGH₂ production in the presence of purified COX-1 enzyme and, in a separate assay, in the presence of purified COX-2 enzyme. In this assay, the production of PGH₂ can be measured by reducing PGH₂ to prostaglandin F_{2α} (PGF_{2α}) with SnCl₂ and then detecting PGF_{2α} by enzyme immunoassay (EIA) using a suitable antibody.

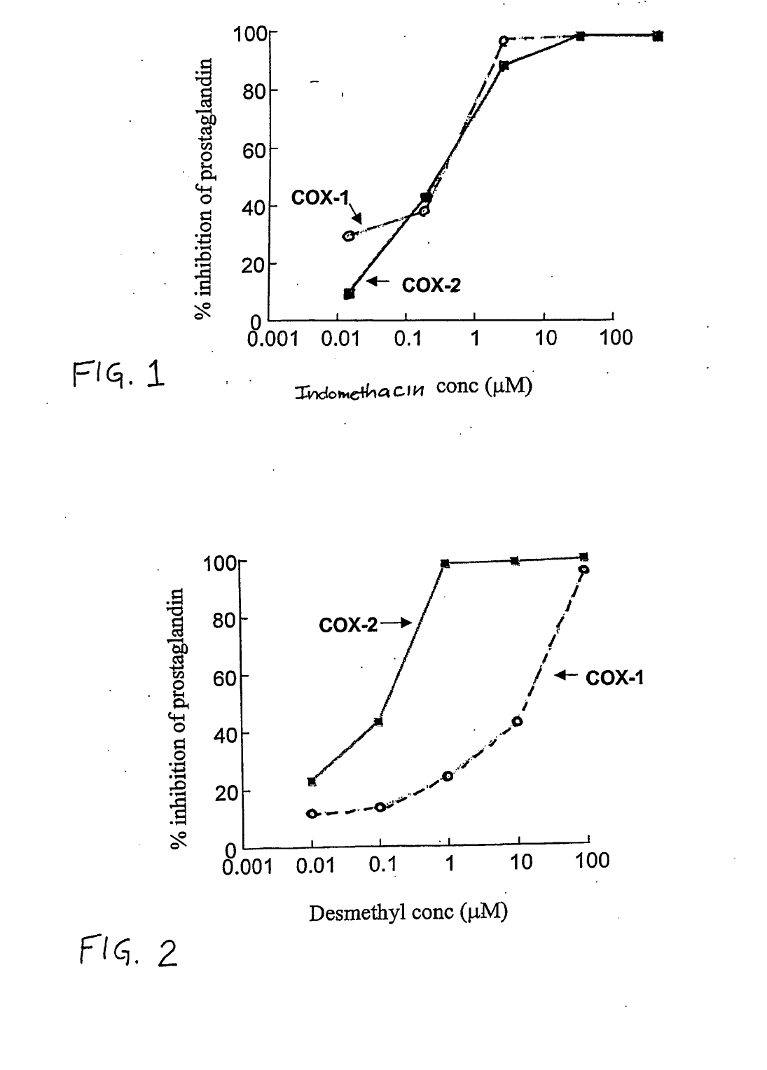

Using the methods described above, the inhibition of human COX-2 and ovine COX-1 by indomethacin was measured. As shown in FIG. 1, the IC₅₀ for inhibition of COX-1 by indomethacin (0.13 μM) was nearly identical to the IC₅₀ for inhibition of COX-2 by indomethacin (0.1 μM) In contrast, the IC₅₀ for inhibition of COX-1 by desmethylindomethacin (15 μM) was 50- to 150-fold greater than the IC₅₀ for inhibition of COX-2 by desmethylindomethacin (0.1 to 0.3 μM. Thus, the COX-2 selectivity (IC₅₀ for COX-1/IC₅₀ for COX-2) of indomethacin is only 1.3, whereas the COX-2 selectivity of desmethylindomethacin is 50-150.

Using the methods described above, the inhibition of human COX-2 and ovine COX-1 by desbenzoylindomethacin was measured. The IC₅₀ for inhibition of COX-1 by desbenzoylindomethacin (20 μM) was nearly identical to the IC₅₀ for inhibition of COX-2 by desbenzoylindomethacin (12 μM). Thus, the COX-2 selectivity of desbenzoylindomethacin is only 1.6, and its potency is reduced nearly 100-fold relative to indomethacin.

### EXAMPLE 2

### Whole Blood COX-1 and COX-2 Assays

A human whole blood assay can also be used to measure the inhibitory activity of compounds on COX-1 and COX-2. Briefly, human whole blood is drawn from 3-6 healthy volunteers who have not taken NSAIDS the previous 2 weeks. To measure COX-1 activity in whole blood, 100 μl of whole blood is combined with a 2 μl aliquot of test compound in vehicle or vehicle alone and incubated for 1 hr at 37° C. as described by Berg et al. (1999 Inflamm. Res. 48, 369-379). Serum is isolated from the sample by centrifugation at 12,000 g for 5 min at 4° C. and then assayed for thromboxane B2 (TXB2) levels using an ELISA assay (e.g., Cayman EIA Kit, Catalog Number 519031). To measure COX-2 activity in whole blood, 100 μl of heparinized whole blood is combined with a 1 μl aliquot of 10 mg/ml LPS (lipopolysaccharide) and a 2 μl aliquot of test compound in vehicle or vehicle alone and incubated for 24 h at 37° C. as described by Berg et al. (supra). Serum is isolated from the sample by centrifugation at 12,000 g for 5 min at 4° C. and assayed for prostaglandin E₂ (PGE₂) using an ELISA assay (e.g., Cayman EIA Kit, Catalog Number 514010).

### EXAMPLE 3

### Measurement of FAAH Activity

The ability of a compound to inhibit FAAH activity can be measured in human whole cell and human and rat brain homogenates as described below.

FAAH Rat Brain Membrane (RBM) Homogenate Preparation

Nine adult rats (Charles River CD strain, female, 200 g) are anaesthetized with isofluorane and rapidly decapitated, respectively. Each brain is quickly removed and chilled in tubes (3 brains per tube) on ice. Total wet weight of the 9 brains is determined. For each ˜18 g of brain, 25 mL of “homogenization buffer” (20 mM HEPES buffer, pH 7.0, with 1 mM MgCl₂) is added to each tube. The brains are homogenized on ice for 1 minute using an Omni GLH homogenizer (Omni International, Marietta, Ga.). The homogenates are transferred to three centrifuge tubes and centrifuged at 36,500 g for 20 minutes at 4° C. The supernatant is discarded and each pellet is re-suspended in 25 ml “homogenization buffer”. The re-suspended material is again centrifuged (36,500 g, for 20 min at 4° C.). Pellets are combined by resuspension in 10 mL of “homogenization buffer” and incubated in a 37° C. water bath for 15 min. The tubes are then placed on ice for 5 min followed by centrifugation at 36,500 g for 20 minutes at 4° C. The supernatant is discarded and the membrane pellets are then re-suspended in 40 mL of “resuspension buffer” (50 mM Tris-HCl buffer, pH 7.4, containing 1 mM EDTA and 3 mM MgCl₂). A Bradford Protein assay is performed to determine protein concentration. The protein is aliquotted into screw cap Cryo tubes each containing ˜400 μL, flash frozen in liquid nitrogen and stored at −80° C. until used for the assay.

FAAH Human Brain Membrane (HBM) Homogenate Preparation

Normal human brain cortex tissue (ca 10 g, pooled from n=3 donors) collected several post mortem and is flash frozen and stored at −80° C. The brain tissue is thawed and transferred to a large ceramic mortar on ice. Fifty mL of ice-cold “homogenization buffer” (20 mM HEPES buffer, pH 7.0, with 1 mM MgCl₂) is added to the mortar and the tissue is homogenized with a pestle. The homogenate is centrifuged at 36,500 g for 20 minutes at 4° C. The supernatants are discarded and the pellets re-suspended in “homogenization buffer” and centrifuged as before. The supernatants are again discarded and the pellets are resuspended in 30 ml homogenization buffer and incubated in a 37° C. water bath for 20 min. The homogenate is then centrifuged as before. The supernatant is discarded and the membrane pellets are re-suspended in 30 ml “resuspension buffer” (50 mM Tris-HCl buffer, pH 7.4, containing 1 mM EDTA and 3 mM MgCl₂). A Bradford Protein assay is performed to determine protein concentration. The protein is aliquotted into screw cap Cryo tubes each containing ˜200 μL, flash frozen in liquid nitrogen and stored at −80° C. until used for the assay.

FAAH Human Carcinoma Cell Membrane (HCM) Homogenate Preparation

Human breast epithelial carcinoma MCF7 cells are obtained from the American Type Culture Collection (ATCC Number HTB-22, Manassas, Va.) and cultured as essentially as described by ATCC. Briefly, cells are grown in Eagle's Minimum Essential Medium (ATCC catalog no. 30-2003) supplemented with 4 mM L-glutamine, 10% final volume of fetal bovine serum (ATCC catalog no. 30-2020), and 0.1 mg/ml human recombinant insulin (Sigma, St. Louis, Mo.). The cells are grown in 5% carbon dioxide in air. When cells reach ˜80% confluency, adherent cells are rinsed with Hanks Balanced Salts Solution (ATCC catalog no. 30-2213), scraped into suspension and collected by centrifugation in a clinical centrifuge at room temperature. Cell pellets are then washed by resuspension in Hanks Balanced Salts Solution followed by centrifugation. Cell pellets are then flash frozen in a dry ice and ethanol bath and stored at −80° C. Cell pellets are thawed and 25 ml of homogenization buffer is added. Membrane homogenates of MCF7 cells are then prepared as described above for rat brain homogenates. A Bradford Protein assay is performed to determine the protein concentration. The protein is aliquotted into screw cap Cryo tubes each containing ˜200 mL, flash frozen in liquid nitrogen and stored at −80° C. until used for the assay.

Determination of FAAH Activity

FAAH activity is assayed in the respective homogenates (Rat brain, Human brain, or Human breast cell carcinoma MCF7 cell) using a modification of the method of Omeir et al. (1995 Life Sci 56:1999) and Fowler et al (1997 J. Pharmacol Exp Ther 283:729). For assay of FAAH in rat brain membrane homogenates (RBM), RBM homogenates (7 μg protein in 20 μl final volume of 10 mM Tris pH 6.5) are mixed with 180 μl of a mixture of the following: 2.0 μM unlabelled anandamide. 0.03 μCi radiolabeled anandamide [ethanolamine 1-³H] (40-60 Ci/mmol, product number ART-626, American Radiolabelled Chemicals, St. Louis, Mo.), 1 mg/ml Bovine Serum Albumin (fatty acid-free BSA, electrophoresis grade, Sigma, St. Louis Mo.), 10 mM Tris-HCl (pH 6.5), and 1 mM EDTA in the presence and absence of inhibitors (vehicle is DMSO at a final concentration of 1%) and incubated for 10 min at 37° C. Samples are placed on ice to terminate the reactions. ³H-ethanolamine product and un-reacted ³H-anandamide substrate are separated by either of 2 respective methods (1) using chloroform methanol extraction or (2) by passing the reaction mixture through a glass fiber filter containing activated charcoal. Samples are extracted with chloroform/methanol by adding 0.4 ml of chloroform/methanol (1:1 v/v), vigorously mixing the samples, and separation of the aqueous and organic phases by centrifugation. Radioactivity (corresponding to FAAH-catalyzed breakdown of ³H-anandamide) found in aliquots (0.2 ml) of the aqueous phase is determined by liquid scintillation counting with quench correction. IC₅₀ values are determined as described by Jonsson et al. (2001 Br J Pharmacol 133:1263). Alternatively, reactions are purified using a modification of the solid-phase extraction method described by Wilson et al (2003 Anal Biochem 318: 270). This method is modified as follows: after reactions are incubated at 37° C. for 10 min and chilled on ice, the reaction mixtures are acidified by adding 10 μl of sodium phosphate solution [0.5M (pH 2.0)]. 90 μl aliquots of the acidified reaction mixtures are applied to activated charcoal (that is previously washed with methanol as described by Wilson et al.) containing 80 μl of water on top of a glass fiber filter, centrifuged, and the radioactivity in the eluate is counted as described previously by Wilson et al.

Synthesis Methods

Useful methods for synthesizing compounds of Formula I wherein R₂ is B can be found in U.S. Pat. No. 5,604,253. Useful methods for synthesizing compounds of Formula I wherein R₂ is C can be found in WO 99/37467. Useful methods for methods for synthesizing compounds of Formula I wherein R₂ is A can be found in WO 96/37468, U.S. Pat. No. 5,436,265 and U.S. Pat. No. 5,510,368.

Preparation of (1-Benzoyl-5-hydroxy-2-methyl-1H-indol-3-yl)acetic acid (1-Benzoyl-5-hydroxy-2-methyl-1H-indol-3-yl)acetic acid was prepared as follows.

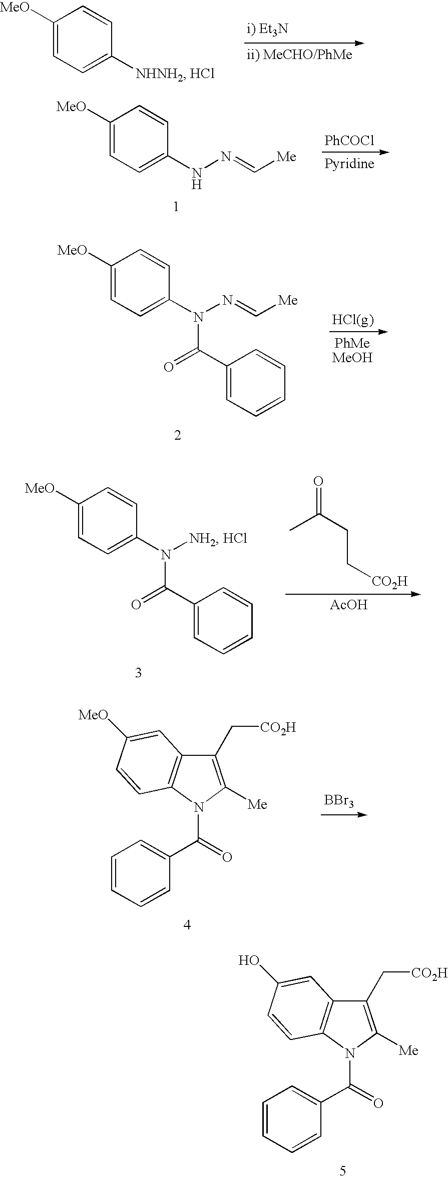

Preparation of (1E)-acetaldehyde (4-methoxyphenyl)hydrazone and (1Z)-acetaldehyde (4-methoxyphenyl)hydrazone (1) Triethylamine (45 mL, 32.7 g, 0.323 mol) was added dropwise to a stirred suspension of 1-(4-methoxyphenyl)hydrazine hydrochloride (60 g, 0.344 mol) in toluene (400 mL), the mixture was stirred at ambient temperature for 30 min, then it was filtered and dried (MgSO₄). The drying agent was removed by filtration, the stirred solution was cooled to 0° C. and acetaldehyde (29 mL, 22.77 g, 0.517 mol) was added dropwise. The mixture was allowed to warm to ambient temperature then it was stirred under nitrogen for 3 h, filtered through Celite, and the solvent was removed in vacuo. The resulting black oil was subjected to Kugelröhr distillation to give (1E)-acetaldehyde (4-methoxyphenyl)hydrazone and (1Z)-acetaldehyde (4-methoxyphenyl)hydrazone (22 g, 39%) as a yellow oil, b.p. 190° C. at 0.1 mmHg. 250 MHz ¹H-NMR (CDCl₃) δ; 1.75 (2×d, 3H, N═CHCH₃, E and Z isomers), 3.64, 3.65 (2×s, 3H, OCH₃, E and Z isomer), 6.55 (q, 0.5H, N═CHCH₃, one isomer), 6.70-6.98 (m, 5.5H, C₆H₄, NH and N═CHCH₃, one isomer).

Preparation of N′-[(1E)-ethylidene]-N-(4-methoxyphenyl)benzohydrazide and N′-[(1Z)-ethylidene]-N-(4-methoxyphenyl)benzohydrazide (2) Benzoyl chloride (28.7 mL, 34.76 g, 0.247 mol) was added dropwise at 10° C. under nitrogen to a stirred solution of (1E)-acetaldehyde (4-methoxyphenyl)hydrazone and (1Z)-acetaldehyde (4-methoxyphenyl)hydrazone (22 g, 0.134 mol) in pyridine (70 mL), the mixture was stirred at ambient temperature for 2 h, and then it was quenched with water (200 mL). The product was extracted into dichloromethane (2×200 mL), the combined extracts were washed with water (2×100 mL), dried (MgSO₄), and the solvent was removed in vacuo. The residual black oil was purified by chromatography over silica using a 30:70 mixture of ethyl acetate and hexane as eluant. Appropriate fractions were combined and the solvents were removed in vacuo to give N′-[(1E)-ethylidene]-N-(4-methoxyphenyl)benzohydrazide and N′-[(1Z)-ethylidene]-N-(4-methoxyphenyl)benzohydrazide (21 g, 58%) as a yellow solid, mp 68-70° C. 250 MHz ¹H-NMR (CDCl₃) δ; 1.80 (d, 3H, N═CHCH₃), 3.75 (s, 3H, OCH₃), 6.79 (q, 1H, N═CHCH₃), 6.90-8.10 (m, 9H, C₆H₄ and Ph).

Preparation of N-(4-methoxyphenyl)benzohydrazide hydrochloride (3) Hydrogen chloride gas was bubbled through a solution of N′-[(1E)-ethylidene]-N-(4-methoxyphenyl)benzohydrazide and N′-[(1Z)-ethylidene]-N-(4-methoxyphenyl)benzohydrazide (21 g, 78.3 mmol) in a mixture of toluene (200 mL) and methanol (50 mL) at 0° C. for one h. The solvents were removed in vacuo and the residual solid was triturated with ethyl acetate (200 mL). The resulting solid was collected by filtration and dried in vacuo to give N-(4-methoxyphenyl)benzohydrazide hydrochloride (12.7 g, 58%) as an off white solid, mp 170-172° C. 250 MHz ¹H-NMR (d₆-DMSO) δ; 3.81 (s, 3H, OCH₃), 7.00 (d, 2H, 2H in Ph), 7.32-7.55 (m, 7H, C₆H₄ and 3H in Ph).

Preparation of (1-benzoyl-5-methoxy-2-methyl-1H-indol-3-yl)acetic acid (4) Levulinic acid (6.34 g, 54.6 mmol) was added to a stirred solution of N-(4-methoxyphenyl)benzohydrazide hydrochloride (12.7 g, 45.6 mmol) in acetic acid (100 ml), the mixture was heated at 80° C. under nitrogen for 3 hours, then it was allowed to cool to ambient temperature and poured onto ice-water (200 mL). The resulting precipitate was collected by filtration and dried in vacuo to give (1-benzoyl-5-methoxy-2-methyl-1H-indol-3-yl)acetic acid (12.7 g, 86%) as a grey solid, mp 158-160° C. 250 MHz ¹H-NMR (CDCl₃) δ; 2.31 (s, 3H, CH₃), 3.60 (s, 2H, CH₂COOH), 3.79 (s, 3H, OCH₃), 6.58 (dd, 1H, indole 6-H), 6.79 (d, 1H, indole 7-H), 6.90 (d, 1H, indole 4-H), 7.40-7.68 (m, 5H, C₆H₅); 10.00-12.00 (very br, cannot be integrated, COOH).

Preparation of (1-benzoyl-5-hydroxy-2-methyl-1H-indol-3-yl)acetic acid (5) Boron tribromide (1 M solution in dichloromethane; 64.9 mL, 64.9 mmol) was added dropwise at −78° C. under nitrogen to a stirred solution of (1-benzoyl-5-methoxy-2-methyl-1H-indol-3-yl)acetic acid (7.0 g, 21.6 mmol) in dichloromethane (200 mL), the mixture was allowed to warm to ambient temperature and stirred for one hour, then it was poured onto water (200 mL). The resulting solid was collected by filtration and dried in vacuo to give (1-benzoyl-5-hydroxy-2-methyl-1H-indol-3-yl)acetic acid (5.6 g, 83%) as a grey solid, mp 186-188° C. 250 MHz ¹H-NMR (d₆-DMSO) δ; 2.12 (s, 3H, CH₃), 3.51 (s, 2H, CH₂COOH), 6.42 (dd, 1H, indole 6-H), 6.75 (m, 2H, indole 4-H and 7-H), 7.42-7.66 (m, 5H, C₆H₅), 9.10 (br, 1H, OH), 12.31 (br, 1H, COOH).

Preparation of (1-Benzoyl-6-fluoro-5-hydroxy-2-methyl-1H-indol-3-yl)acetic acid (1-Benzoyl-6-fluoro-5-hydroxy-2-methyl-1H-indol-3-yl)acetic acid was prepared as follows.

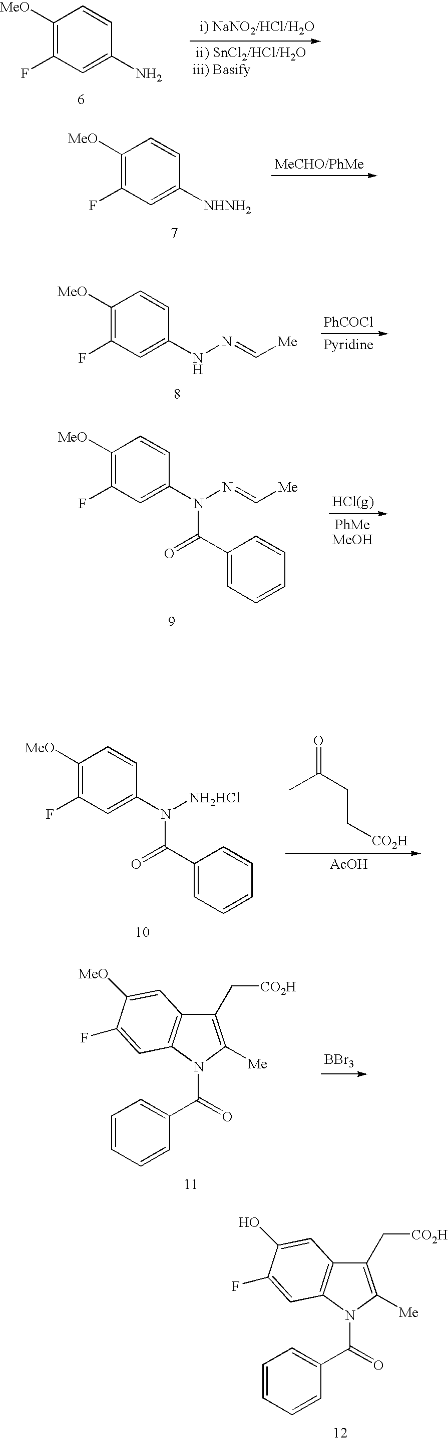

Preparation of (3-fluoro-4-methoxyphenyl)hydrazine (7) 3-Fluoro-4-methoxyaniline (6) (95 g, 0.67 mol) was added to concentrated hydrochloric acid (250 mL), the suspension was stirred at ambient temperature for 18 h, then it was cooled to 0° C. and a solution of sodium nitrite (53.7 g, 0.78 mol) in water (200 mL) was added dropwise at 0-5° C. When the addition was complete, the resulting solution was stirred at 0° C. for 1 h then it was added dropwise at 0-5° C. to a stirred solution of tin(II) chloride dihydrate (638.9 g, 2.83 mol) in concentrated hydrochloric acid (500 mL). The mixture was allowed to warm to ambient temperature then it was stored at 4° C. for 18 h. The resulting precipitate was collected by filtration, washed with water (400 mL), and ether (1000 mL) and dried in vacuo. The solid hydrochloride salt was basified by addition to 10% aqueous sodium hydroxide solution (800 mL), the free base was extracted into ether (2×400 mL), and the combined extracts were dried (MgSO₄) and the solvent removed in vacuo to give (3-fluoro-4-methoxyphenyl)hydrazine (51.9 g, 50%) as a yellow solid, mp 46-50° C. 250 MHz ¹H-NMR (CDCl₃) δ (ppm): 1.5 (s, 1H, NH—NH₂), 3.85 (s, 3H, OCH₃), 5.0 (s, 2H, NH—NH₂), 6.44 (m, 1H, phenyl 6-H), 6.60 (dd, 1H, phenyl 5-H), 6.79 (t, 1H, phenyl 2-H).

Preparation of (1E)-acetaldehyde (3-fluoro-4-methoxyphenyl)hydrazone and (1Z)-acetaldehyde (3-fluoro-4-methoxyphenyl)hydrazone (8) Acetaldehyde (11.2 mL, 8.8 g, 0.2 mol) was added dropwise at 0° C. under nitrogen to a stirred mixture of (3-fluoro-4-methoxyphenyl)hydrazine (20.8 g, 0.133 mol), magnesium sulfate (80 g) and toluene (200 mL), the mixture was stirred at ambient temperature for 3 h, then it was filtered and the solvent was removed in vacuo. The residue was subjected to Kugelröhr distillation to give (1E)-acetaldehyde (3-fluoro-4-methoxyphenyl)hydrazone and (1Z)-acetaldehyde (3-fluoro-4-methoxyphenyl)hydrazone (10.5 g, 43%) as a yellow oil, bp 190° C. at 1 mmHg, which solidified slowly at ambient temperature. This material was used in the next step without further purification.

Preparation of N′-[(1E)-ethylidene]-N-(3-fluoro-4-methoxyphenyl) benzohydrazide (9) Benzoyl chloride (14.0 mL, 16.95 g, 0.121 mol) was added dropwise at 10° C. under nitrogen to a stirred solution of (1E)-acetaldehyde (3-fluoro-4-methoxyphenyl)hydrazone and (1Z)-acetaldehyde (3-fluoro-4-methoxyphenyl)hydrazone (11 g, 0.06 mol) in pyridine (100 mL), the mixture was stirred at ambient temperature for 2 hours, then it was added to ice-water (250 mL). The product was extracted into dichloromethane (2×200 mL), the combined extracts were dried (MgSO₄) and the solvent was removed in vacuo. The residue was purified by flash chromatography over silica using a 30:70 mixture of ethyl acetate and hexane as eluant. Appropriate fractions were combined and the solvents were removed in vacuo to give N′-ethylidene-N-(3-fluoro-4-methoxyphenyl)benzohydrazide (8 g, 47%) as a yellow solid, mp 110-112° C. 250 MHz ¹H-NMR (CDCl₃) δ; 1.85 (d, 3H, N═CHCH₃), 3.85 (s, 3H, OCH₃), 6.8 (q, 1H, N═CHCH₃), 6.90-7.7 (m, 8H, C₆H₃ and Ph).

Preparation of N-(3-fluoro-4-methoxyphenyl)benzohydrazide hydrochloride (10) Hydrogen chloride gas was bubbled through a stirred solution of N′-ethylidene-N-(3-fluoro-methoxyphenyl)benzohydrazide (8 g, 0.028 mol) in a mixture of toluene (200 mL) and methanol (10 mL) at 0° C. for 1 h. The solvents were removed in vacuo, the residue was triturated with ethyl acetate (150 mL) and the resulting solid was collected by filtration and dried in vacuo to give N-(3-fluoro-4-methoxyphenyl)benzohydrazide hydrochloride (5 g, 60%) as a white solid, mp 175-178° C. 250 MHz ¹H-NMR (d₆-DMSO) δ; 3.9 (s, 3H, OCH₃), 7.1-7.55 (m, 8H, C₆H₃ and Ph), 8-10 (very br, 3H, —NH₃⁺).

Preparation of (1-benzoyl-6-fluoro-5-methoxy-2-methyl-1H-indol-3-yl)acetic acid (11) A stirred mixture of N-(3-fluoro-4-methoxyphenyl)benzohydrazide hydrochloride (4.7 g, 15.8 mmol), levulinic acid (2.2 g, 19 mmol) and acetic acid (20 mL) was heated at 80° C. for 4 h, cooled to ambient temperature and added to ice-water (50 mL). The resulting solid was collected by filtration, dried in vacuo and crystallised from toluene to give a 5:1 mixture of (1-benzoyl-6-fluoro-5-methoxy-2-methyl-1H-indol-3-yl)acetic acid and (1-benzoyl-4-fluoro-5-methoxy-2-methyl-1H-indol-3-yl)acetic acid (3.6 g) as a grey solid. A portion (2.6 g) of the mixture was purified by column chromatography over silica using a 1:1 mixture of ethyl acetate and hexane containing a few drops of acetic acid as eluant. Appropriate fractions were combined and the solvents were removed in vacuo to give a small sample (200 mg) of (1-benzoyl-6-fluoro-5-methoxy-2-methyl-1H-indol-3-yl)acetic acid, and a larger sample (1.6 g) which was still a mixture of the two regioisomers. The larger sample (1.6 g) was subjected to a further purification by UV-triggered radial compression Biotage chromatography using gradient elution with 15:85-30:70 mixtures of ethyl acetate and hexane as eluants. Appropriate fractions were combined and the solvents were removed in vacuo to give (1-benzoyl-6-fluoro-5-methoxy-2-methyl-1H-indol-3-yl)acetic acid (500 mg) and a larger sample (1.1 g) which was a mixture of 11 and (1-benzoyl-4-fluoro-5-methoxy-2-methyl-1H-indol-3-yl)acetic acid. (1-Benzoyl-6-fluoro-5-methoxy-2-methyl-1H-indol-3-yl)acetic acid (0.7 g, 13%) was thus obtained as a white solid, mp 118-120° C. 250 MHz ¹H-NMR (CDCl₃) δ; 2.30 (s, 3H, CH₃), 3.61 (s, 2H, CH₂CO₂H), 3.88 (s, 3H, OCH₃), 6.71 (d, 1H, indole 4-H), 6.90 (d, 1H, indole 7-H), 7.48 (m, 2H, 2H in Ph), 7.59 (m, 3H, 3H in Ph), 10.00-11.00 (very br. COOH).

Preparation of (1-benzoyl-6-fluoro-5-hydroxy-2-methyl-1-indol-3-yl)acetic acid (12) Boron tribromide (1 M solution in dichloromethane; 3.51 mL, 3.51 mmol) was added dropwise at −78° C. under nitrogen to a stirred solution of (1-benzoyl-6-fluoro-5-methoxy-2-methyl-1H-indol-3-yl)acetic acid (400 mg, 1.17 mmol) in dichloromethane (10 mL), the mixture was allowed to warm to ambient temperature, then it was stirred for a further 4.5 h. The mixture was added to ice-water (15 mL) and the resulting solid was collected by filtration and dried in vacuo to give (1-benzoyl-6-fluoro-5-hydroxy-2-methyl-1H-indol-3-yl)acetic acid (200 mg, 52%) as a white solid, mp 159-161° C. 250 MHz ¹H-NMR (d₆-DMSO) δ; 2.12 (s, 3H, CH₃), 3.59 (s, 2H, CH₂CO₂H)), 6.81 (d, 1H, indole 4-H), 7.01 (d, 1H, indole 7-H), 7.66 (m, 5H, C₆H₅), 9.69 (s, 1H, OH), 12.41 (s, 1H, COOH).

Animal Models for Testing Anti-Inflammatory and Analgesic Activity of Compounds

Any of a variety of animal models can be used to test the compounds of the invention for their effectiveness in reducing inflammation and treating pain. Useful compounds can exhibit effectiveness in reducing inflammation or pain in one or animal models.

Animal Models for Assessing Anti-Inflammatory Activity

Collagen Arthritis Model

Autoimmunity to type H collagen can be used as an experimental model of arthritis. In this model, rats are injected intradermally with type II collagen extracted from human, chick or rat cartilage in combination with complete Freund's adjuvant or incomplete Freund's adjuvant. This induces inflammatory arthritis in approximately 40% of treated rats. The disease induced is a chronic proliferative synovitis, resembling adjuvant arthritis in rats and rheumatoid arthritis in humans (Trentham et al. 1977 J Exp Med 146:857).

Carrageenan-Induced Foot Pad Edema Model

The model is described, for example, by Winter et al. (Proc Soc Exp. Biol Med 111:544 1962). Briefly, rats are fasted with free access to water for 17 to 19 hours before oral treatment with up to three doses of a test compound, indomethacin or celecoxib, or a control vehicle (1% methylcellulose in deionized water). One hour after the last treatment, paw edema is induced by injecting 0.05 ml of a 2% carrageenan solution into the left hindpaw.) The left hindpaw volume of each rat is measured using a plethysmometer before oral treatment, at the time of carrageenan injection and at 1.5 h, 3 h, 4.5 h after the injection of carrageenan. The edema volume of each rat at each time point is expressed as the change from the volume at the time of oral treatment and the anti-inflammatory effect in treated groups is expressed as % inhibition compared to the vehicle only group 1.5 h, 3 h and 4.5 h after the carrageenan injection. The significance of the difference between in edema different groups is assessed by a one-way analysis of variance (ANOVA) followed by the non-paired Dunnett t test. In this model, hyperalgesic response and PGE₂ production can also be measured (Zhang et al. 1997 J Pharmacol and Exp Therap 283:1069).

Complete Freund's Adjuvant (CPA) Induced Arthritis Model

Briefly, arthritis is induced in groups of eight Lewis derived male rats weighing 160±10 g by injecting a well-ground suspension of killed Mycobacterium tuberculosis (0.3 mg in 0.1 ml of light mineral oil; Complete Freund's Adjuvant, CFA) into the subplantar region of the right hind paw on Day 1. Hind paw volumes are measured by water displacement on Days 0, 1 and 5 (right hind paw, with CFA), and on Days 0, 14 and 18 (left hind paw, without CFA); rats are weighed on Days 0 and 18. Test compounds, dissolved or suspended in 2% Tween 80, are prepared fresh daily and administered orally twice daily for 5 consecutive days (Day 1 through day 5) beginning one hour before injection of CFA. For CFA-injected vehicle control rats, the increase in paw volume on Day 5 relative to Day 1 (Acute Phase of inflammation) is generally between 0.7 and 0.9 ml; that on Day 18 relative to day 14 (Delayed Phase of inflammation) is generally between 0.2 and 0.4 ml. Thus, anti-inflammatory activity in this model may be denoted by values calculated during the Acute Phase as well as the Delayed Phase. Animals are also weighed on Day 0 and Day 18; CFA-injected vehicle control animals generally gain between 40 to 60 g body weight over this time period. A 30 percent or more reduction in paw volume relative to vehicle treated controls is considered of significant anti-inflammatory activity. The mean ±SEM for each treatment group is determined and Dunnett test is applied for comparison between vehicle and treated groups. Differences are considered significant at P<0.05. Polyarthritis of fore paw, tail, nose and ear can be scored visual y and noted on the first day and final day, wherein positive (+) sign is for swelling response and negative (−) sign is normal. X-ray radiographies of the hindpaws can also be performed for further radiological index determination of arthritic symptoms. Hyperalgesia can also be measured in this model, allowing determination of analgesic effects of test compounds Bertorelli et al. 1999 Brit Journ Pharmacol 128:1252)

Air-Pouch Model

This model is described by Masferrer et al. (1994 Proc Natl Acad Sci USA 91:3228). Briefly, male Lewis rats (175-200 g, Harlan Sprague-Dawley) are subcutaneously injected with 20 ml of sterile air into the intrascapular area of the back to create air cavities. An additional 10 ml of air is injected into the cavity every 3-days to keep the space open. Seven days after the initial air is injection, 2 ml of a 1% solution of carrageenan dissolved in sterile saline is injected directly into the pouch to produce an inflammatory response. In treated and untreated animals the volume of exudate is measured and the number of leukocytes present in the exudate is determined by Wright-Giemsa staining. In addition, PGE₂ and 6-keto-PGF_{1α} are determined in the pouch exudates from treated and untreated animals by specific ELISAs (Cayman Chemicals, Ann Arbor, Mich.).

Animal Models for Assessing Pain Control Activity

Carreageenan-Induced Thermal Hyperalgesia

This model is described by Hargreaves et al. (1988 Pain 32:77-88). Briefly, inflammation is induced by subplantar injection of a 2% carrageenan suspension (0.1 ml) into the right hindpaw. Three hours later, the nociceptive threshold is evaluated using a thermal nociceptive stimulation (plantar test). A light beam (44% of the maximal intensity) is focused beneath the hindpaw and the thermal nociceptive threshold is evaluated by the paw flick reaction latency (cut-off time: 30 sec). The pain threshold is measured in ipsilateral (inflamed) and in contralateral (control) hindpaws, 1 hour after the oral treatment with the test compound or a control. The results can be expressed as the nociceptive threshold in seconds (sec) for each hindpaw and the percentage of variation of the nociceptive threshold (mean ±SEM) for each rat from the mean value of the vehicle group. A comparison of the nociceptive threshold between the inflamed paw and the control paw of the vehicle-treated group is performed using a Student's t test, a statistically significant difference is considered for P<0.05. Statistical significance between the treated groups and the vehicle group is determined by a Dunnett's test using the residual variance after a one-way analysis of variance (P<0.05) using SigmaStat Software.

Phenylbenzoquinone-Induced Writhing Model

This model is described by Siegmund et al. (1957 Proc. Soc. Exp. Bio. Med. 95:729-731). Briefly, one hour after oral dosing with a test compound, morphine or vehicle, 0.02% phenylbenzoquinone (PBQ) solution (12.5 mL/kg) is injected by intraperitoneal route into the mouse. The number of stretches and writhings are recorded from the 5th to the 10th minute after PBQ injection, and can also be counted between the 35^{th} and 40^{th} minute and between the 60^{th} and 65^{th} minute to provide a kinetic assessment. The results are expressed as the number of stretches and writhings (mean ±SEM) and the percentage of variation of the nociceptive threshold calculated from the mean value of the vehicle-treated group. The statistical significance of any differences between the treated groups and the control group is determined by a Dunnett's test using the residual variance after a one-way analysis of variance (P<0.05) using SigmaStat Software.

Kaolin-Induced Arthritis Model.

This model is described by Hertz et al. (1980 Arzneim. Forsch 30:549-1557). Briefly, arthritis is induced by injection of 0.1 ml of kaolin suspension into the knee joint of the right hind leg of a rat. Test compounds are administered subcutaneously after 15 minutes and again after two hours. Reference compounds can be administered orally or subcutaneously. Gait is assessed every hour from 1.5 hours to 5.5 hours after treatment and is scored as follows: normal gait (0), mid disability (1), intermittent raising of paw (2), and elevated paw (3). Results are expressed as the mean gait score (mean ±SEM) calculated from individual values at each time point and the percentage of variation of the mean score calculated from the mean value of the vehicle-treated group at 4.5 hours and 5.5 hours after treatment. The statistical significance of differences between the treated groups and the vehicle-treated group is determined by a Dunnett's test using the residual variance after a one-way analysis of variance (P<0.05) at each time point.

Peripheral Mononeuropathy Model

This model is described by Bennett et al. (Pain 33:87-107, 1988) and can be used to assess anti-hyperalgesic effect of an orally administered test compound in a model of peripheral mononeuropathy. The effect of the test substance can be compared to a no treatment control or reference substance, e.g., morphine. Peripheral mononeuropathy is be induced by loose ligation of the sciatic nerve in anaesthetized male Sprague Dawley rats (pentobarbital; 45 mg/kg by intraperitoneal route). Fourteen days later, the nociceptive threshold is evaluated using a mechanical nociceptive stimulation (analgesimeter paw pressure test; Ugo Basile, Italy). The test and reference compounds and the vehicle are orally administered (10 ml/kg carried 1% methylcellulose). Increasing pressure is applied to the hindpaw of the animal until the nociceptive reaction (vocalization or paw withdrawal) is reached. The pain threshold (grams of contact pressure) is measured in ipsilateral (injured) and in contralateral (non injured) hindpaws, 60 minutes after treatment. The results are expressed as: the nociceptive threshold (mean ±SEM) in grams of contact pressure for the injured paw and for the non-injured paw (vehicle-treated group) and the percentage of variation the nociceptive threshold calculated from the mean value of the vehicle-treated group. A comparison of the nociceptive threshold between the non injured paw and the injured paw of the vehicle-treated group is performed using a Student's t test. The statistical significance of the difference between the treated groups and the vehicle group is determined for the injured hindpaw by a Dunnett's test using the residual variance after a one-way analysis of variance (P<0.05) using SigmaStat Software (SigmaStat® v. 2.0.3 (SPSS Science Software, Erkrath GmbH)).

Diabetic Neuropathy Paw Pressure Test

Complete protocol details can be found in Rakieten et al. (1963 Cancer Chemother. Rep. 29:91). Briefly, diabetes is induced by intraperitoneal injection of streptozotocin in rats. Three weeks later, the nociceptive threshold is measured using the paw pressure test to assess hyperalgesia Test compound or controls are administered intraperitoneally 30 minutes prior to pain measurement.

Acetic Acid Writhing Test

Briefly, a test compound is administered orally one hour before intraperitoneal injection of acetic acid (0.5%, 10 ml/kg) in rats. Reduction in the number of writhes by 50 percent or more (50) per group of animals observed during the 5 to 11 minute period after acetic acid administration, relative to a vehicle treated control group, indicates possible analgesic activity. This assay is based on that described in Inoue, K. et al. (1991 Arzneim. Forsch./Drug Res. 41: 235).

Formalin Test

Complete protocol details can be found in Hunskaar et al. (1985 Neurosci. Meth. 14:69). Briefly, 30 minutes after intraperitoneal-administration of a test compound or a control, 20 μl of a 5% formalin solution is injected by subplantar route into the right hindpaw of the rat. Hindpaw licking time is recorded during the early phase and the later phase after formalin injection.

Tail Flick Test

Complete protocol details can be found in D'Amour and Smith (1941 J. Pharmacol. Exp Ther. 72:74). Briefly, 30 minutes after intraperitoneal administration of a test compound or a control, a light beam is focused onto the tail of the rat. The nociceptive reaction latency, characterized by tail withdrawal, is recorded. The cutoff time is set to 15 seconds.

Tail Immersion Test

In this test the tail of the rat is immersed into a 50-60° C. water bath. The nociceptive reaction latency, characterized by tail withdrawal, is measured (Haubrich et al. 1990 J Pharmacol Exp Ther 255:511 and Lichtman et al. 2004 Pain 109:319).

Hot Plate Test

Complete protocol details can be found in Eddy et al. (1950 J Pharmacol. Exp. Ther. 98:121). Briefly, 30 minutes after intraperitoneal administration of a test compound or a control, the mouse is placed on a metallic hot plate maintained at 52° C. The nociceptive reaction latency, characterized by a licking reflex of the forepaws or by a jumping off the hot plate is recorded. The cut-off time is set to 30 seconds.

Assays for Assessing Anxiolytic Activity

Compounds of the invention that modulate FAAH activity, and thus fatty acid amide levels, may also have anxiolytic activity. Animal models to assess anxiolytic activity include those described below.

Elevated Plus Maze

The elevated plus maze consists of four maze arms that originate from a central platform, effectively forming a plus sign shape as described in van Gaalen and Steckler (2000 Behavioural Brain Research 115:95). The maze can be made of plexiglas and is generally elevated. Two of the maze arms are unwalled (open) and two are walled (closed). The two open arms are well lit and the two enclosed arms are dark (Crawley 2000 What's Wrong With My Mouse?: Behavioral Phenotyping of Transgenic and Knockout Mice. Wiley-Liss, New York). The test is premised on the naturalistic conflict between the tendency of an animal to explore a novel environment and the aversive properties of a brightly lit, open area (Pellow et al. 1985 J. Neuroscience Methods. 14:149).

Complete protocol details can be found in Fedorova et al. (2001 J. Pharm. Exp. Ther. 299: 332). Briefly, 15 minutes following intraperitoneal administration of test compound or control, an animal is placed individually on the central platform, facing one of the open-arms opposite to the observer. The number of open and closed arm entries, and the time spent in the different compartments of the maze by the animal (central platform, open and closed arms) is scored (as described in Gaalen et al. (supra)). An arm visit is recorded when an animal moves all four paws into the arm as described in Simonin et al. (1998 EMBO J. 17: 886). Behavior is scored by an observer and/or via a video camera over a 5-minute test session. A greater amount of time spent or entries made by the animal in the open versus the closed arms is an indicator of anxiolytic activity.

Elevated Zero Maze

The elevated zero maze is a modification of the elevated plus maze. The elevated zero maze consists of a plexiglas apparatus in the shape of a circle (i.e., a circular runway of 46 cm diameter and 5.5 cm runway width) with two open and two wall-enclosed sectors of equal size. It is elevated up to a meter above the ground. This apparatus is described in Simonin et al. (supra) and Crawley (supra).

Complete protocol details can be found in Kathuria et al (2003 Nature Medicine 9:76). Briefly, 30 minutes following intraperitoneal administration of test compound or control, an animal is placed on one open sector in front of an enclosed sector. Time in a new sector is recorded as entry with all four paws. Behavior will be scored by an observer and/or via a video camera over a 5-minute test session. A greater amount of time spent or entries made by the animal in the open versus the walled sector is an indicator of anxiolytic activity.

Assays for Assessing Anti-Nociception Mechanism

Compounds can be tested to determine if they influence pathways involved in nociception. The results of such assays can be used to investigate the mechanism by which a test compound mediates its anti-nociceptive effect.

Elevation of 3α,5α-THP

3α-hydroxy-5α-pregan-20-one (3α,5α-THP or allopregnanolone) is a pregnane steroid that acts as an agonist of the inhibitory GABA_{A} receptor subtype and is known to have both anxiolytic and analgesic effects in a variety of animal systems, with supportive evidence for a similar role in humans. Thus, compounds that elevate 3α,5α-THP may have an antinociceptive effect. The level of 3α,5α-THP in the brain of animals treated with a test compound can be measured as described by VanDoren et al. (J. Neuroscience 20:200, 1982) as follows. Briefly, steroids are extracted from individual cerebral cortical hemispheres dissected in ice-cold saline after euthanasia. Cortices are frozen at −80° C. until use. Samples are digested in 0.3 N NaOH by sonication and extracted three times in 3 ml aliquots of 10% (v/v) ethyl acetate in heptane. The aliquots are combined and diluted with 4 ml of heptane. The extracts are applied to solid phase silica columns (Burdick & Jackson, Muskegon, Mich.), washed with pentane, and steroids of similar polarity to 3α,5α-THP are eluted off of the column by the addition of 25% (v/v) acetone in pentane. The eluant is then dried under N₂ and steroids are redissolved in 20% (v/v) isopropanol RIA buffer (0.1 M NaH₂PO₄, 0.9 M NaCl, 0.1% w/v BSA, pH 7.0). Extraction efficiency is determined in 50 μl of the redissolved extract by liquid scintillation spectroscopy and the remaining sample is used in the determination of 3α,5α-THP by radioimmunoassay. Reconstituted sample extracts (75 μl) and 3α,5α-THP standards (5-40,000 pg in 6.25% v/v ethanol, 31% v/v isopropyl alcohol in RIA buffer) are assayed in duplicate by the addition of 725 μl of RIA buffer, 100 μl of [³H]3α,5α-THP (20,000 dpm), and 100 μl of anti-3α,5α-THP antibody. Total binding is determined in the absence of unlabeled 3α,5α-THP, and nonspecific binding is determined in the absence of antibody. The antibody-binding reaction is allowed to equilibrate for 120 min at room temperature and is terminated by cooling the mixture to 4° C. Bound 3α,5α-THP is separated from unbound 3α,5α-THP by incubation with 300 μl of cold dextran coated charcoal (DCC; 0.04% dextran, 0.4% powdered charcoal in double-distilled H₂O) for 20 min. DCC is removed by centrifugation at 2000×g for 10 min. Bound radioactivity in the supernatant is determined by liquid scintillation spectroscopy. Sample values are compared to a concurrently run 3α,5α-THP standard curve and corrected for extraction efficiency.

Inhibition of Fatty Acid Amidohydrolase

The endogenous cannabinoid system is involved in the regulation of nociception, among other physiological effects. One component of this system is fatty acid amide hydrolase (FAA), which inactivates the fatty acid amide anandamide. Inhibitors of FAAH could thus inhibit anandamide degradation and result in increased levels of anandamide, with resulting analgesic effects. The effect of test, compounds on FAAH activity can be assayed in human whole cell and in human and rat brain homogenates as described above.

Whole Cell Anandamide Hydrolysis Assay

FAAH activity can be assayed in whole cells using methods disclosed previously (Maccarone et al. 1998 J Biol Chem 273:32332 and Bisogno et al. 1997 J Biol Chem 272:3315). In addition to the cell lines described in Maccarone et al. and Bisogno et al., MCF7 (ATCC designation HTB-22) and T84 (ATCC designation CCL-248) cell lines may be used in these assays.

Determination of Endogenous and Exogenous Anandamide Levels in Rat Plasma and Brain Tissue

The effects of test compounds on endogenous and exogenously dosed anandamide levels can be measured. Rats dosed with test article are, sacrificed at various time points to determine the levels of anandamide both circulating and within the brain tissue. For experiments measuring exogenous levels of anandamide, the anandamide (Cayman Chemical, Ann Arbor, Mich. or Sigma Chemical, St. Louis, Mo.) is dosed (in the range of 3-30 mg/kg) intraperitoneally (IP) 30 minutes post dosing of test compound. Animals are sacrificed at either 15, 30, or 60 minutes after anandamide administration upon anesthesia administration followed by decapitation. Brains are immediately extracted and the plasma is recovered from the blood.

Anandamide is extracted from the plasma by first precipitating the proteins by adding an equal volume of cold acetone with 10 ng of d8-anadamide (Cayman Chemicals, Ann Arbor, Mich.) as an internal standard. The acetone is evaporated from the supernatant followed by an extraction with chloroform:methanol (2:1). The chloroform layer is collected and evaporated to dryness. The pellet containing the anandamide is resuspended into methanol:chloroform (3:1) and injected onto an Xterra IS 2.1×20 mm C8 column (Waters Corporation, Milford, Mass.) and followed by detection by a Waters Quattro Micro LCMSMS (Waters Corporation, Milford, Mass.). The HPLC method consists of a step gradient (mobile phase A: 10 mM ammonium hydroxide in water, mobile phase B: 20% methanol in Acetonitrile) starting at 25% B and stepping up to 90% B at 2.2 minutes and holding for 2 minutes. Quantities are measured against known standards spiked into blank plasma using MassLynx v.4.0 software (Waters Corporation, Milford, Mass.).

Levels of anandamide from brain tissue are determined as follows. Brain tissue is homogenized in ethyl acetate and water (3:1) with 10 ng of d8-anadamide (Cayman Chemicals, Ann Arbor, Mich.) as an internal standard. The ethyl acetate layer is collected and evaporated to dryness. The pellet containing anandamide is resuspended in methanol:chloroform (3:1) and analyzed by the same method as plasma and normalized against the fresh tissue weight.

Cannabinoid Receptor Binding

Compounds may exert an antinociceptive effect via binding to either or both of the cannabinoid receptors CB₁ and CB₂. CB₁ is expressed in the brain (Matsuda et al. 1990 Nature 346:561-564), and CB₂ is expressed by macrophages and in the spleen (Munro et al. 1993 Nature 365:61-65). Both of these receptors have been implicated in mediating analgesic effects through binding of agonists (see, for example, Clayton, N. et al., 2002 Pain, 96(3):253-60). Thus, test compounds can be assayed to determine whether they bind to one or both human cannabinoid receptors. An assay for CB₁ binding is described by Matsuda et al. (supra). This assay employs recombinant cells expressing CB₁. Binding to CB₂ can be determined in the same manner using recombinant cells expressing CB₂. Briefly, to measure the ability of a test compound to bind to CB₁, the binding of a labelled CB₁ ligand, e.g., [³H]WIN 55212-2 (2 nM for CB₁ and 0.8 nM for CB₂) to membranes are isolated from HEK-293 cells expressing recombinant CB₁ is measured in the presence and absence of a compound. Non-specific binding is separately determined in the presence of several-fold excess of unlabelled WIN 55212-2 (5 μM for CB₁ and 10 μμ for CB₂). The specific ligand binding to the receptors is defined as the difference between the total binding and the non-specific binding determined in the presence of an excess of unlabelled WIN 55212-2. The IC₅₀ values and Hill coefficients (n_{H}) are determined by non-linear regression analysis of the competition curves using Hill equation curve fitting. The inhibition constants (Kᵢ) are calculated from the Cheng Prusoff equation (Kᵢ=IC₅₀/(1+(L/K_{D})), where L=concentration of radioligand in the assay, and K_{D}=affinity of the radioligand for the receptor).

Therapeutic Methods

The compounds of the invention can be used, for example, to treat conditions or disorders in which it is considered desirable to reduce or eliminate COX-2 activity and/or FAAH activity. Thus, they can be used in any situation in which a COX-2 inhibitor or FAAH inhibitor is used as well as in other situations. For example, compounds of Formula I, Formula II and IIa and related prodrugs can be used to treat an inflammatory disorder, including both disorders in which inflammation is considered a significant component of the disorder and those in which, inflammation is considered a relatively minor component of the disorder, to treat acute and chronic pain (analgesic) and to treat fever (antipyretic). Among the inflammatory disorders that can be treated are auto-immune disorders.

Disorders that can be treated with a composition comprising a compound having Formula I, Formula II and IIa and related prodrugs include: arthritis (including rheumatoid arthritis, spondyloarthopathies, gouty arthritis, degenerative joint diseases (e.g., osteoarthritis), systemic lupus erythematosus, ankylosing spondylitis, acute painful shoulder, psoriatic, and juvenile arthritis), asthma, atherosclerosis, osteoporosis, bronchitis, tendonitis, bursitis, skin inflammation disorders (e.g., psoriasis, eczema, burns, dermatitis), enuresis, eosinophilic disease, gastrointestinal disorders (including inflammatory bowel disease, peptic ulcers, regional enteritis, diverticulitis, gastrointestinal bleeding, Crohn's disease, gastritis, irritable bowel syndrome and ulcerative colitis), and disorders ameliorated by a gastroprokinetic agent (e.g., ileus, for example post-operative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP)).

The compounds of the invention can also be used in the treatment of symptoms associated with influenza or other viral infections, common cold, sprains and strains, myositis, neuralgia, synovitis, injuries such as sports injuries and those following surgical and dental procedures, coagulation disorders, kidney disease (e.g., impaired renal function), ophthalmic disorders (including glaucoma, retinitis, retinopathies, uveitis and acute injury to the eye tissue), liver diseases (e.g., inflammatory liver disease including chronic viral hepatitis B, chronic viral hepatitis C, alcoholic liver injury, primary biliary cirrhosis, autoimmune hepatitis, nonalcoholic steatohepatitis and liver transplant rejection), and pulmonary inflammatory diseases (e.g., asthma, allergic rhinitis, respiratory distress syndrome chronic bronchitis, and emphysema). Compositions comprising a compound having Formula I, Formula II and IIa and related prodrugs can also be used to treat, for example, inflammation associated with: vascular diseases, migraine headaches, tension headaches, periarteritis nodosa, thyroiditis, aplastic anemia, Hodgkin's disease, sclerodoma, rheumatic fever, type I diabetes, myasthenia gravis, sarcoidosis, nephrotic syndrome, Behcet's syndrome, polymyositis, gingivitis, hypersensitivity, conjunctivitis, multiple sclerosis, and ischemia (e.g., myocardial ischemia), and the like. The compounds may be useful for treating neuroinflammation associated with brain disorders (e.g., Parkinson's disease and Alzheimer's disease) and chronic inflammation associated with cranial radiation injury. The compounds may be useful for treating acute inflammatory conditions (such as those resulting from infection) and chronic inflammatory conditions (such as those resulting from asthma, arthritis and inflammatory bowel disease). The compounds may also be useful in treating inflammation associated with trauma and non-inflammatory myalgia. The compounds can also be administered to those prior to surgery or taking anticoagulants. The compounds of the invention may reduce the risk of a thrombotic cardiovascular event which is defined as any sudden event of a type known to be caused by platelet aggregation, thrombosis, and subsequent ischemic clinical events, including thrombotic or thromboembolic stroke, myocardial ischemia, myocardial infarction, angina pectoris, transient ischemic attack (TIA; amaurosis fagax), reversible ischemic neurologic deficits, and any similar thrombotic event in any vascular bed (splanchnic, renal, aortic, peripheral, etc.).

The compounds of the invention may inhibit uterus contraction caused by hormones and prostanoid-induced smooth muscle contraction. The compounds of the invention may be useful in treating premature labor, menstrual cramps, menstrual irregularity, and dysmenorrhea.

The compounds of the invention may inhibit cellular neoplastic transformations and metastatic tumor growth. The compounds of the invention may be associated with reducing the number of adenomatous colorectal polyps. Thus, compounds and prodrugs may also be useful in reducing the risk of certain cancers, e.g., solid tumor cancers such as colon or colorectal cancer. The compounds and prodrugs may also be used in the treatment of prevention of all cancers including cancers of the bladder, cancers associated with overexpression of HER-2/neu cervix, skin, esophagus, head and neck, lung including non small-cell lung cancers, kidney, pancreas, prostate, gall bladder and bile duct and endometrial cancers, gastric cancers, gliomas, hepatocellular carcinomas, colonic adenomas, mammary cancers, ovarian cancers and salivary cancers. In addition, the compounds and prodrugs may be useful in treating large intestine cancer and prostate cancer. The compounds may also be useful in cases where the patient is at risk for cancer including oral premalignant lesions, cervical intraepithelial neoplasia, chronic hepatitis, bile duct hyperplasia, atypical adenomatous hyperplasia of lung, prostatic, intraepithelial neoplasia, bladder dysplasia, actinic keratoses of skin, colorectal adenomas, gastric metaplasia, and Barrett's esophagus.

Compounds of the invention are also useful for the treatment of cognitive disorders such as dementia, particularly degenerative dementia (including senile dementia, Alzheimer's disease (and precursors thereof), Pick's disease, Huntington's chorea, Parkinson's disease and Creutzfeldt-Jakob disease), and vascular dementia (including multiinfarct dementia), as well as dementia associated with intracranial space occupying lesions, trauma, infections and related conditions (including HIV infection), metabolism, toxins, anoxia and vitamin deficiency; and mild cognitive impairment associated with ageing, particularly Age Associated Memory Impairment.

Compounds of the invention may also prevent neuronal injury by inhibiting the generation of neuronal free radicals (and hence oxidative stress) and therefore are of use in the treatment of stroke; epilepsy; and epileptic seizures (including grand mal, petit mal, myoclonic epilepsy and partial seizures). The compounds of the invention may be useful to control or suppress seizures (including those that are chemically induced).

The compounds of the invention can be used in treatment of all varieties of pain including pain associated with a cough condition, pain associated with cancer, preoperative pain, arthritic pain and other forms of chronic pain such as post-operative pain, lumbosacral pain, musculo-skeletal pain, headache, migraine, muscle ache, lower back and neck pain, toothache and the like. The compounds of the invention are also useful for the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy, neuralgia, such as post-herpetic neuralgia and trigeminal neuralgia; and pain resulting from physical trauma, amputation, cancer, toxins or chronic inflammatory conditions. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The compounds of the invention may also be of use in the treatment and/or prevention of cyclooxygenase-mediated proliferative disorders such as may occur in diabetic retinopathy and tumor angiogenesis. The compounds of the invention may be used to inhibit angiogenesis, such as occurs in wet macular degeneration.

The compounds of the invention may also be used for treating sexual behavior problems and/or improving sexual performances.

The compounds useful in the prevention and/or treatment of pain, in particular acute or chronic neurogenic pain, migraine, neuropathic pains including the forms associated with herpes virus and diabetes, acute or chronic pain associated with the inflammatory diseases: arthritis, rheumatoid arthritis, osteoarthritis, spondylitis, gout, vascularitis, Crohn's disease; irritable bowel syndrome and acute/sharp or chronic pains at the periphery. The compounds of the invention can also be used to prevent and/or treat emesis, dizziness, vomiting, and nausea, especially after chemotherapy, food behavioral problems/feeding disorders (e.g., eating disorders, in particular anorexias and cachexias of various natures, weight loss associated with cancer and other wasting conditions), neurological pathologies, psychiatric tremors (e.g., dyskinesias, dystonia, spasticity, obsessive compulsive behavior, Tourette's syndrome, all forms of depression and anxiety of any nature and origin, mood disturbances, psychoses), acute or chronic neurodegenerative diseases (e.g., Parkinson's disease, Alzheimer's disease, senile insanity, Huntington's chorea, lesions related to cerebral ischemia and cranial and medullary traumas, epilepsy, sleep disorders (sleep apnea), cardiovascular diseases (in particular hypertension, cardiac arrhythmias, arteriosclerosis, heart attacks, cardiac ischemias, renal ischemia), cancers (benign tumors of the skin, papillomas and cerebral tumors, prostate tumors, cerebral tumors (glioblastomas, medullary epitheliomas, medullary blastomas, neuroblastomas, tumors of origin, astrocytomas, astroblastomas, ependymomas, oligodendrogliomas, plexus tumor, neuroepithelioma, epiphysis tumor, ependyblastomas, malignant meningiomas, sarcomatosis, malignant melanomas, schwan cell cancers), disorders of the immune system (in particular autoimmune diseases including psoriasis, erythematous lupus), diseases of conjunctive or connective tissue, Sjogren's syndrome, spondylarthritis anchylosis, undifferentiated spondylarthritis undifferentiated, Behcet's disease, autoimmune hemolytic anaemias, multiple sclerosis, amyotrophic side sclerosis, amyloses, graft rejection, and illnesses affecting the blastocytes, allergic diseases (e.g., immediate or delayed hypersensitivity, allergic rhinitis or conjunctivitis, contact dermatitis), viral or bacterial parasitic infectious diseases (i.e. AIDS, meningitis), inflammatory diseases (in particular arthritic diseases such as: arthritis, rheumatoid arthritis osteoarthritis, spondylitis, gout, vascularitis, Crohn's disease, irritable bowel syndrome, osteoporosis, psoriasis, ocular infections and disorders (e.g., ocular hypertension, glaucoma, wet macular degeneration), lung diseases (e.g., diseases of the respiratory tracts, bronchyospasms, cough, asthma, chronic bronchitis, chronic obstruction of the respiratory tracts, emphysema), gastrointestinal disorders (e.g., irritable bowel syndrome, intestinal inflammatory disorders, ulcers, diarrheas, acid reflux), urinary incontinence, vesical inflammation, movement disorders, psychomotor disorders, hypertension, and AIDS-related complex. The compounds of the invention can be used as a sleep aid, to treat insomnia or to induce sleep. The compounds may be used to reduce dr control body weight (or fat) or prevent and/or treat obesity or other appetite related disorders related to the excess consumption of food, ethanol and other appetizing substances. The compounds may be used to modulate lipid metabolism, reduce body fat (e.g., via increasing fat utilization) or reduce (or suppress) appetite (e.g., via inducing satiety). The compounds of the invention may be used to prevent, control or treat schizophrenia, paranoia or other related disorders, or other disorders of dopamine transmission.

The compounds of the invention can also be used to treat anxiety (including generalized anxiety disorder, panic disorder, and social anxiety Disorder) and depression.

Administration of Compounds

The compounds of the invention can be used alone or in combination with other compounds used to treat inflammatory disorders. Combination therapies are useful in a variety of situations, including where an effective dose of one or more of the agents used in the combination therapy is associated with undesirable toxicity or side effects when not used in combination. This is because a combination therapy can be used to reduce the required dosage or duration of administration of the individual agents.

Thus, the compounds of the invention can be used in a co-therapy with a second agent, e.g., an anti-inflammatory agent. Anti-inflammatory agents which can be used in co-therapy include: NSAIDs, 5-lipoxygenase(LO) inhibitors (e.g., masoprocol, tenidap, zileuton, pranlukast, tepoxalin, rilopirox, and flezelastine hydrochloride, enazadrem phosphate, and bunaprolast), p38 inhibitors (e.g. SB203580 and Vertex compound VX745), LTB₄ antagonists and LTA₄ hydrolase inhibitors, CRTH2 modulators (e.g. ramatroban), steroids, corticosteroids (e.g. betamethasone, budesonide, cortisone, prednisone, triamcinolone, methylprednisone, prednisone, and dexamethasone, hydrocortisone), Bayer Bay-x-1005, Ciba Geigy compound CGS-25019C, Leo Denmark compound ETH-615, Lilly compound LY-293111, Ono compound ONO-4057, Terumo compound TMK-688, Lilly compounds LY-213024, 264086 and 292728, ONO compound ONO-LB457, Searle compound SC-53228, calcitrol, Lilly compounds LY-210073, LY-223982, LY-233469, and LY-255283, ONO compound ONO-LB448, Searle compounds SC41930, SC-50605 and SC-51146, and SmithKline SKF-104493.

The compounds of the invention can be used in combination with selective COX-2 inhibitors, e.g., Celecoxib®, Valdecoxib®, Parecoxib®, Rofecoxib®, Etoricoxib®, and Lumaricoxib®.

The compounds of the invention can be used in a co-therapy with a an agent used to treat an anxiety disorders, including: benzodiazepines (e.g., Xanax®, Librium®) and SSRIs (e.g., Prozac®, Zoloft®), monoamine oxidase inhibitors (MAOIs) and tricyclic antidepressants (TCAs, e.g., amitryptilline).

The compounds of the invention can be used in a co-therapy with a an agent used to treat rheumatoid arthritis including etanercept (Enbrel®) and infliximab (Remicade®).

The compounds of the invention can also be used in a co-therapy with a second agent that has analgesic activity. Analgesics which can be used in co-therapy include, but are not limited to: NSAIDs (e.g., aspirin, ibuprofen, fenoprofen, acetaminophen, phenacetin, diclofenac, etodolac, ketoprofen, ketorolac, flurbiprofen, indomethacin, mefenamic acid, diflusinal, fenbufen, meclofenamic acid, sulindac, flufenisal, piroxicam, phenylbutazone, tolmetin, zomepirac, nabumetone oxaprozin and naproxen), a non-narcotic analgesic such as tramadol, or a narcotic analgesic (e.g., codeine, oxycodone; dihydrocodeine, hydrocodone, loperamide, fedotozine, and fentanyl, naloxone, naltrexone, methyl nalozone, nalmefene, cypridime, beta funaltrexamine, naloxonazine, naltrindole, and nor-binaltorphimine, morphine, diphenyloxylate, enkephalin pentapeptide, and trimebutine), levorphanol, APF112, mepivacaine, ), NK1 receptor antagonists (e.g., ezlopitant and SR-14033, SSR-241585), CCK receptor agonists (e.g., loxiglumide), NK1 receptor antagonists, NK3 receptor antagonists (e.g., talnetant, osanetant SR-142801, SSR-241585), norepinephrine-serotonin reuptake inhibitors (NSRI; e.g., milnacipran), vanilloid receptor agonists and antagonists, cannabinoid receptor agonists (e.g., arvanil), sialorphin, compounds or peptides that are inhibitors of neptilysin, frakefamide (H-Tyr-D-Ala-Phe(F)-Phe-NH₂; WO 01/019849 A1), loperamide, Tyr-Arg (kyotorphin), CCK receptor agonists (caerulein), conotoxin peptides, peptide analogs of thymulin, loxiglumide, dexloxiglumide (the R-isomer of loxiglumide) (WO 88/05774), and analgesic peptides (e.g. endomorphin-1, endomorphin-2, nocistatin, dalargin, lupron, and substance P). In addition, certain antidepressants can be used in co-therapy either because they have analgesic activity or are otherwise beneficial to use in combination with an analgesic. Examples of such anti-depressants include: selective serotonin reuptake inhibitors (e.g., fluoxetine, paroxetine, sertraline), serotonin-norepinephrine dual uptake inhibitors, venlafaxine and nefazadone. Certain anti-convulsants have analgesic activity and are useful in co-therapy. Such anti-convulsants include: gabapentin, carbamazepine, phenyloin, valproate, clonazepam, topiramate and lamotrigine. Such agents are considered particularly useful for treatment of neuropathic pain, e.g., treatment of trigeminal neuralgia, postherpetic neuralgia, and painful diabetic neuropathy. Additional compounds useful in co-therapy include: alpha-2-adrenergic receptor agonists (e.g., tizanidine and clonidine), mexiletine, corticosteroids, compounds that block the NMDA (N-methyl-Daspartate) receptor (e.g, dextromethorphan, ketamine, and amantadine), glycine antagonists, carisoprodol, cyclobenzaprine, various opiates, nonopioid antitussive such as dextromethorphan, carmiphen, cararniphen or carbetapentane, opioid antitussives such as codeine or hydrocodone and metaxolone. The compounds of the invention can also be combined with inhalable gaseous nitric oxide (for treating pulmonary vasoconstriction or airway constriction), a thromboxane A2 receptor antagonist, a stimulant (i.e. caffeine), an H₂-antagonist (e.g. ranitidine), an antacid (i.e. aluminum or magnesium hydroxide), an antiflatulent (i.e. simethicone), a decongestant (including phenylephrine, phenylpropanolamine, pseudophedrine, oxymetazoline, ephinephrine, naphazoline, xylometazoline, propylhexedrine, or levodesoxyephedrine), a prostaglandin (i.e. misoprostol, enprostil, rioprostil, ornoprostol or rosaprostol), a diuretic, a sedating or non-sedating antihistamine, a 5HT1 agonist, such as a triptan (e.g. sumatriptan or naratriptan), an adenosine Al agonist, an EP ligand, a sodium channel blocker (e.g. lamotrigine), a substance P antagonist (e.g. an NK, antagonist), a cannabinoid, a 5-lipoxygenase inhibitor, a leukotriene receptor antagonist, a DMARD (e.g. methotrexate), a neurone stabilising antiepileptic drug, a mono-aminergic uptake inhibitor (e.g. venlafaxine), a matrix metalloproteinase inhibitor, a nitric oxide synthase (NOS) inhibitor, such as an iNOS or an nNOS inhibitor, an inhibitor of the release, or action, of tumor necrosis factor, an antibody therapy, such as a monoclonal antibody therapy, an antiviral agent, such as a nucleoside inhibitor (e.g. lamivudine) or an immune system modulator (e.g. interferon), a local anaesthetic, a known FAAH inhibitor (e.g., PMSF, URB532, URB597, or BMS-1, as well as those described in those described in WO04033652, U.S. Pat. No. 6,462,054, US20030092734, US20020188009, US20030195226, and WO04033422), an antidepressant (e.g., VPI-013), a fatty acid amide (e.g. anandamide, N-palmitoyl ethanolamine, N-oleoyl ethanolamide, 2-arachidonoylglycerol, or oleamide), arvanil, analogs of anadamide and arvanil as described in US20040122089, and a proton pump inhibitor (e.g., omeprazole).

The compound of the invention can also be used in a co-therapy with a second agent that is a cannabanoid receptor antagonist to prevent and/or treat obesity and other appetite related disorders.

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

The agents, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques.

The agent can be in the form of a pharmaceutically acceptable salt. Such salts are prepared from pharmaceutically acceptable non-toxic bases including inorganic bases and organic bases. Examples of salts derived from inorganic bases include aluminum, ammonium, calcium, copper, ferric, ferrous, lithium, magnesium, manganic salts, manganous, potassium, sodium, zinc, and the like. In some embodiments, the salt can be an ammonium, calcium, magnesium, potassium, or sodium salt. Examples of salts derived from pharmaceutically acceptable organic non-toxic bases include salts of primary, secondary, and tertiary amines, benethamine, N,N′-dibenzylethylenediamine, diethylamine, 2-diethylaminoethanol, 2-dimethylaminoethanol, diethanolamine, ethanolamine, ethylenediamine, N-ethylmorpholine, N-ethylpiperidine, epolamine, glucamine, glucosamine, histidine, hydrabamine, isopropylamine, lysine, methylglucamine, meglumine, morpholine, piperazine, piperidine, polyamine resins, procaine, purines, theobromine, triethylamine, trimethylamine, tripropylamine, and trolamine, tromethamine. Examples of other salts include arecoline, arginine, barium, betaine, bismuth, chloroprocaine, choline, clemizole, deanol, imidazole, and morpholineethanol. In one embodiment are tris salts.

The agents of the invention can be administered orally, e.g., as a tablet or cachet containing a predetermined amount of the active ingredient, pellet, gel, paste, syrup, bolus, electuary, slurry, capsule; powder; granules; as a solution or a suspension in an aqueous liquid or a non-aqueous liquid; as an oil-in-water liquid emulsion or a water-in-oil liquid emulsion, via a liposomal formulation (see, e.g., EP 736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents of the invention can also be administered by captisol delivery technology, rectal suppository or parenterally.

Compositions of the present invention may also optionally include other therapeutic ingredients, anti-caking agents, preservatives, sweetening agents, colorants, flavors, desiccants, plasticizers, dyes, and the like. Any such optional ingredient must be compatible with the compound of the invention to insure the stability of the formulation.

The composition may contain other additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents such as:

BINDERS: corn starch, potato starch, other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch (e.g., STARCH 1500® and STARCH 1500 LM®, sold by Colorcon, Ltd.), hydroxypropyl methyl cellulose, microcrystalline cellulose (e.g. AVICEL™, such as, AVICEL-PH-101™, -103™ and -105™, sold by FMC Corporation, Marcus Hook, Pa., USA), or mixtures thereof,

FILLERS: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, or mixtures thereof,

DISINTEGRANTS: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof,

LUBRICANTS: calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Deaussa Co., Plano, Tex. USA), a pyrogenic silicon dioxide (CAB-O-SIL, Cabot Co., Boston, Mass. USA), or mixtures thereof,

ANTI-CAKING AGENTS: calcium silicate, magnesium silicate, silicon dioxide, colloidal silicon dioxide, talc; or mixtures thereof,

ANTIMICROBIAL AGENTS: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propylparaben, sodium benzoate, sodium dehydroacetate, sodium propionate, sorbic acid, thimersol, thymo, or mixtures thereof, and

COATING AGENTS: sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnauba wax, microcrystalline wax, or mixtures thereof.

The agents either in their free form or as a salt can be combined with a polymer such as polylactic-glycoloic acid (PLGA), poly-(I)-lactic-glycolic-tartaric acid (P(I)LGT) (WO 01/12233), polyglycolic acid (U.S. Pat. No. 3,773,919), polylactic acid (U.S. Pat. No. 4,767,628), poly(ε-caprolactone) and poly(alkylene oxide) (US20030068384) to create a sustained release formulation. Such formulations can be used to implants that release a compound of the invention or another agent over a period of a few days, a few weeks or several months depending on the polymer, the particle size of the polymer, and the size of the implant (see, e.g., U.S. Pat. No. 6,620,422). Other sustained release formulations are described in EP 0 467 389 A2, WO 93/241150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. No. 5,968,895, U.S. Pat. No. 6,180,608, US 20030171296, US 20020176841, U.S. Pat. No. 5,672,659, U.S. Pat. No. 5,893,985, U.S. Pat. No. 5,134,122, U.S. Pat. No. 5,192,741, U.S. Pat. No. 5,192,741, U.S. Pat. No. 4,668,506, U.S. Pat. No. 4,713,244, U.S. Pat. No. 5,445,832 U.S. Pat. No. 4,931,279, U.S. Pat. No. 5,980,945, WO 02/058672, WO 9726015, WO 97/04744, and US20020019446. In such sustained release formulations microparticles of compound are combined with microparticles of polymer. U.S. Pat. No. 6,011,011 and WO 94/06452 describe a sustained release formulation providing either polyethylene glycols (where PEG 300 and PEG 400 are most preferred) or triacetin. WO 03/053401 describes a formulation which may both enhance bioavailability and provide controlled release of the agent within the GI tract. Additional controlled release formulations are described in WO 02/38129, EP 326151, U.S. Pat. No. 5,236,704, WO 02/30398, WO 98/13029; US20030064105, US20030138488A1, US20030216307A1, U.S. Pat. No. 6,667,060, WO 01/49249, WO 01/49311, WO 01/49249, WO 01/49311, and U.S. Pat. No. 5,877,224.

The agents can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), or by other routes. The agents can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g., WO 97/11682) via a liposomal formulation (see, e.g., EP 736299, WO 99/59550 and WO 97/13500), via formulations described in WO 03/094886 or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The agents can also be administered transdermally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al. 2004, Nature Reviews Drug Discovery 3:115)). The agents can be administered using high-velocity transdermal particle injection techniques using the hydrogel particle formulation described in US20020061336. Additional particle formulations are described in WO 00/45792, WO 00/53160, and WO 02/19989. An example of a transdermal formulation containing plaster and the absorption promoter dimethylisosorbide can be found in WO 89/04179. WO 96/11705 provides formulations suitable for transdermal administration. The agents can be administered in the form a suppository or by other vaginal or rectal means. The agents can be administered in a transmembrane formulation as described in WO 90/07923. The agents can be administered non-invasively via the dehydrated particles described in U.S. Pat. No. 6,485,706. The agent can be administered in an enteric-coated drug formulation as described in WO 02/49621. The agents can be administered intranasaly using the formulation described in U.S. Pat. No. 5,179,079. Formulations suitable for parenteral injection are described in WO 00/62759. The agents can be administered using the casein formulation described in US20030206939 and WO 00/06108. The agents can be administered using the particulate formulations described in US20020034536.

The agents, alone or in combination with other suitable components, can be administered by pulmonary route utilizing several techniques including but not limited to intratracheal instillation (delivery of solution into the lungs by syringe), intratracheal delivery of liposomes, insufflation (administration of powder formulation by syringe or any other similar device into the lungs) and aerosol inhalation. Aerosols (e.g., jet or ultrasonic nebulizers, metered-dose inhalers (MDIs), and dry-powder inhalers (DPIs)) can also be used in intranasal applications. Aerosol formulations are stable dispersions or suspensions of solid material and liquid droplets in a gaseous medium and can be placed into pressurized acceptable propellants, such as hydrofluoroalkanes (HFAs, e.g., HFA-134a and HFA-227, or a mixture thereof), dichlorodifluoromethane (or other chlorofluocarbon propellants such as a mixture of Propellants 11, 12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion. Pulmonary formulations may also include surfactants which include but are not limited to bile salts and those described in U.S. Pat. No. 6,524,557 and references therein. The surfactants described in U.S. Pat. No. 6,524,557, e.g., a C8-C16 fatty acid salt, a bile salt, a phospholipid, or alkyl saccharide are advantageous in that some of them also reportedly enhance absorption of the compound in the formulation. Also suitable in the invention are dry powder formulations comprising a therapeutically effective amount of active compound blended with an appropriate carrier and adapted for use in connection with a dry-powder inhaler. Absorption enhancers which can be added to dry powder formulations of the present invention include those described in U.S. Pat. No. 6,632,456. WO 02/080884 describes new methods for the surface modification of powders. Aerosol formulations may include U.S. Pat. No. 5,230,884, U.S. Pat. No. 5,292,499, WO 017/8694, WO 01/78696, US2003019437, US20030165436, and WO 96/40089 (which includes vegetable oil). Sustained release formulations suitable for inhalation are described in US 20010036481A1, US20030232019A1, and US 20040018243A1 as well as in WO 01/13891, WO 02/067902, WO 03/072080, and WO 03/079885. Pulmonary formulations containing microparticles are described in WO 03/015750, US20030008013, and WO 00/00176. Pulmonary formulations containing stable glassy state powder are described in US20020141945 and U.S. Pat. No. 6,309,671. Other aerosol formulations are described in EP1338272A1 WO 90/09781, U.S. Pat. No. 5,348,730, US 6,436,367, WO 91/04011, and U.S. Pat. No. 6,294,153 and U.S. Pat. No. 6,290,987 describes a liposomal based formulation that can be administered via aerosol or other means. Powder formulations for inhalation are described in US20030053960 and WO 01/60341. The agents can be administered intranasally as described in US20010038824.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy. Other devices for generating aerosols employ compressed gases, usually hydrofluorocarbons and chlorofluorocarbons, which are mixed with the medicament and any necessary excipients in a pressurized container, these devices are likewise described in standard textbooks such as Sprowls and Remington.

The agent can be fused to immunoglobulins or albumin, or incorporated into a liposome to improve half-life. The agent can also be conjugated to polyethylene glycol (PEG) chains. Methods for pegylation and additional formulations containing PEG-conjugates (i.e. PEG-based hydrogels, PEG modified liposomes) can be found in Harris and Chess, Nature Reviews Drug Discovery 2: 214-221 and the references therein. The agent can be administered via a nanocochleate or cochleate delivery vehicle (BioDelivery Sciences International). The agents can be delivered transmucosally (i.e. across a mucosal surface such as the vagina, eye or nose) using formulations such as that described in U.S. Pat. No. 5,204,108. The agents can be formulated in microcapsules as described in WO 88/01165. The agent can be administered intra-orally using the formulations described in US20020055496, WO 00/47203, and U.S. Pat. No. 6,495,120. The agent can be delivered using nanoemulsion formulations described, in WO 01/91728A2.

The agents can be a free acid or base, or a pharmacologically acceptable salt thereof. Solids can be dissolved or dispersed immediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means

Suitable pharmaceutical compositions in accordance with the invention will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences, 18th Ed., Mack Publishing Company, 1995.

Methods to increase chemical and/or physical stability of the agents the described herein are found in WO 00/04880, and WO 97/04796 and the references cited therein.

Methods to increase bioavailability of the agents described herein are found in US20030198619, WO 01/49268, WO 00/32172, and WO 02/064166. Glycyrrhizinate can also be used as an absorption enhancer (see, e.g., EP397447). WO 03/004062 discusses Ulex europaeus I (UEAI) and UEAI mimetics which may be used to target the agents of the invention to the GI tract. The agents described herein and combination therapy agents can be packaged as a kit that includes single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation.

A number of embodiments of the invention have been described. Nevertheless, it will be understood that various modifications may be made without departing from the spirit and scope of the invention.

1. A compound having the formula 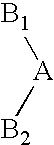
wherein:
A is a 5-membered or 6-membered substituted or unsubstituted heteroaryl or heterocyclyl ring having one or two heteroatoms selected from the group consisting of O, S, and N and having from 1 or 2 independent substituents when the ring is 5-membered and substituted with 1 to 4 independent substituents when the ring is 6-membered and substituted; and
each of B₁ and B₂ is the same or different substituted or unsubstituted 6-membered aryl or heteroaryl ring;
and salts thereof.

2. (canceled)

3. The compound of claim 1 wherein at least one of B₁ and B₂ is singly or independently multiply substituted and the substituents are selected from: a methyl group optionally, independently substituted with one or more halogen, an ethyl group optionally, independently substituted with one or more halogen, a halogen, —OH, —OCH₃, optionally, independently substituted with one or more halogen, and —SCH₃ optionally, independently substituted with one or more halogen.

4. (canceled)

5. The compound of claim 1 wherein A is selected from the group consisting of oxazolyl, isoxazolyl, thiazolyl, isothiazolyl, pyrazolidinyl, pyrazolyl, furanyl, and pyridinyl.

6. The compound of claim 1 wherein each of B₁ and B₂ is a singly or multiply substituted phenyl group.

7. (canceled)

8. The compound of claim 1 wherein one or both of B₁ and B₂ is:

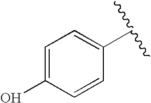

9. The compound of claim 1 selected from the group consisting of: 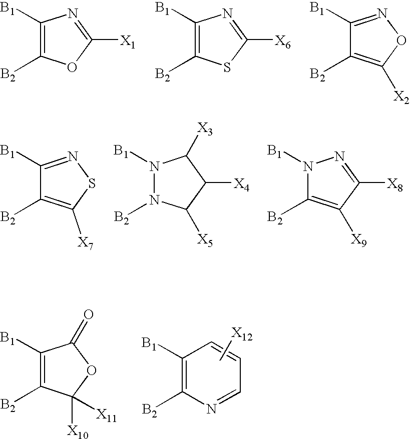
wherein:
each X₁-X₁₂ is independently: H, halogen, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₆ alkoxy, oxo, substituted or unsubstituted C₂₋₁₂ alkenyloxy, substituted or unsubstituted C₅₋₁₀ cycloalkenyloxy, substituted or unsubstituted (C₂₋₁₂ alkynyl)oxy, (C₁₋₆ alkyl)oxy(C₁₋₆ alkyl), substituted or unsubstituted C₆₋₁₂ aryloxy, (C₃₋₆ heteroaryl)-(C₁₋₆ alkyl)oxy, (C₁₋₁₂ alkyl)thio, substituted or unsubstituted (C₁₋₄ alkyl)-thio-(C₁₋₄ alkyl), substituted or unsubstituted C₆-C₁₀ aryl, substituted or unsubstituted styryl, substituted or unsubstituted C₃₋₁₂ heteroaryl, substituted or unsubstituted C₄₋₈ heterocyclic, wherein the substituents are selected from the group consisting of hydroxy, halo, C₁₋₄ alkyl, C₁₋₄ trihaloalkyl, C₁₋₆ alkoxy, C₁₋₄ trihaloalkoxy, bivalent oxy(C₁₋₆)alkyloxy, (C₁₋₆) acylamino, (C₁₋₆) acylthio, amino, and azido; or R⁵ and R⁶ form a C₅-C₁₀ heteroaryl ring, and each of R⁴, R⁷, and R⁸ is, independently, hydroxy, halo, C₁₋₄ alkyl, C₁₋₄ trihaloalkyl, C₁₋₆ alkoxy, or C₁₋₄ trihaloalkoxy.

10-16. (canceled)

17. A compound having the formula: 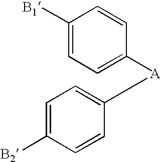
wherein
A is a 5-membered or 6-membered substituted or unsubstituted heteroaryl or heterocyclyl ring having one or two heteroatoms selected from the group consisting of O, S, and N and having from 1 or 2 independent substituents when the ring is 5-membered and substituted and 1 to 4 independent substituents when the ring is 6-membered and substituted; and one or both of B₁ and B₂ are selected from H, —OH, 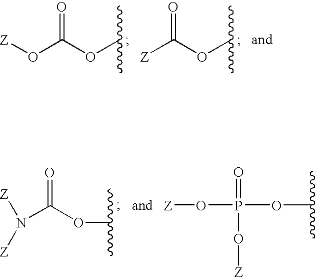
wherein each Z is independently H or a C₁₋₆ straight chain or branched alkyl, alkenyl, alkynyl, aryl, cycloalkyl, or arylalkyl that is optionally singly or multiply substituted; and and salts thereof.

18. The compound of claim 17 wherein A is selected from: 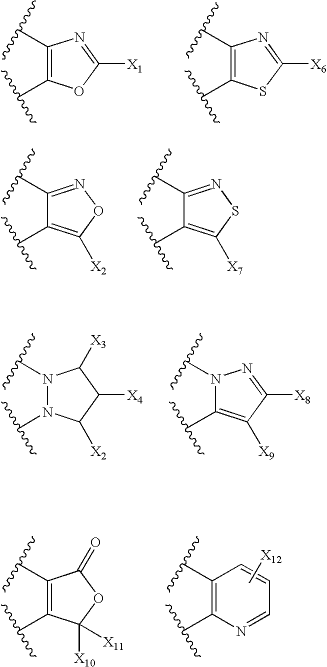
wherein each X₁-X₁₂ is independently: H, halogen, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₆ alkoxy, oxo, substituted or unsubstituted C₂₋₁₂ alkenyloxy, substituted or unsubstituted C₅₋₁₀ cycloalkenyloxy, substituted or unsubstituted (C₂₋₁₂ alkynyl)oxy, (C₁₋₆ alkyl)oxy(C₁₋₆ alkyl), substituted or unsubstituted C₆₋₁₂ aryloxy, (C₃₋₆ heteroaryl)-(C₁₋₆ alkyl)oxy, (C₁₋₁₂ alkyl)thio, substituted or unsubstituted (C₁₋₄ alkyl)-thio-(C₁₋₄ alkyl), substituted or unsubstituted C₆-C₁₀ aryl, substituted or unsubstituted styryl, substituted or unsubstituted C₃₋₁₂ heteroaryl, substituted or unsubstituted C₄₋₈ heterocyclic, wherein the substituents are selected from the group consisting of hydroxy, halo, C₁₋₄ alkyl, C₁₋₄ trihaloalkyl, C₁₋₆ alkoxy, C₁₋₄ trihaloalkoxy, bivalent oxy(C₁₋₆)alkyloxy, (C₁₋₆) acylamino, (C₁₋₆) acylthio, amino, and azido; or R⁵ and R⁶ form a C₅-C₁₀ heteroaryl ring, and each of R⁴, R⁷, and R⁸ is, independently, hydroxy, halo, C₁₋₄ alkyl, C₁₋₄ trihaloalkyl, C₁₋₆ alkoxy, or C₁₋₄ trihaloalkoxy.

19. The compound of claim 18 wherein A is selected from:

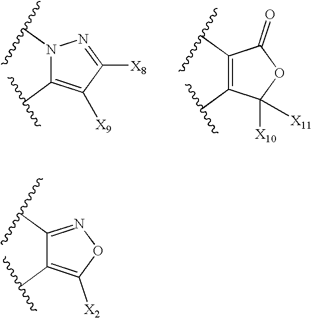

20. The compound of claim 19 wherein X₂ is selected from H, CH₃, COOH, —CH₂—CH₂—COOH, —CH₂—COOH and CF₃; X₁₀ and X₁₁ are H or one or both of X₁₀ and X₁₁ are CH₃ or CF₃; X₉ is missing; and X₈ is CH₃ or CF₃.

21. (canceled)

22. The compound of claim 17 selected from: 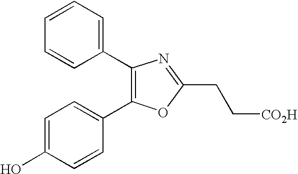
3-[5-(4-hydroxyphenyl)-4-phenyl-1,3-oxazol-2-yl]propionic acid 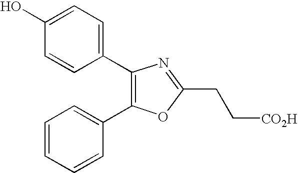
3-[4-(4-hydroxyphenyl)-5-phenyl-1,3-oxazol-2-yl]propionic acid 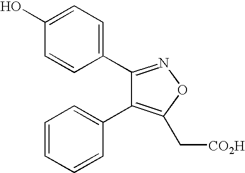
[3-(4-hydroxyphenyl)-4-phenylisoxazol-5-yl]acetic acid 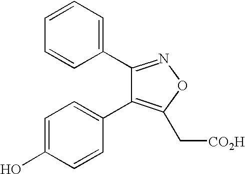
[4-(4-hydroxyphenyl)-3-phenylisoxazol-5-yl]acetic acid 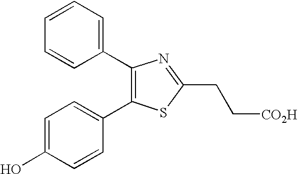
3-[4-(4-hydroxyphenyl)-4-phenyl-1,3-thiazol-2-yl]propionic acid 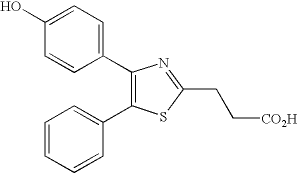
3-[4-(4-hydroxyphenyl)-5-phenyl-1,3-thiazol-2-yl]propionic acid 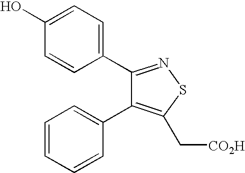
[3-(4-hydroxyphenyl)-4-phenylisothiazol-5-yl]acetic acid 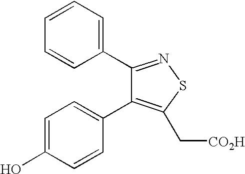
[4-(4-hydroxyphenyl)-3-phenylisothiazol-5-yl]acetic acid 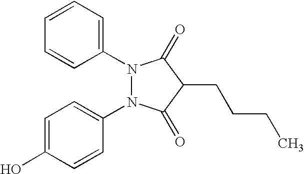
4-butyl-1-(4-hydroxyphenyl)-2-phenylpyrazolidine-3,5-dione 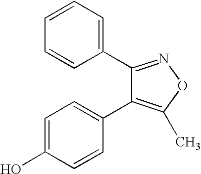
4-(5-methyl-3-phenylisoxazol-4-yl)phenol 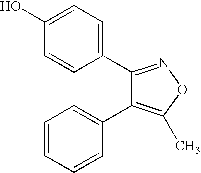
4-(5-methyl-4-phenylisoxazol-3-yl)phenol 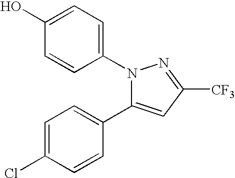
-1-(4-hydroxyphenyl)-3-(trifluoromethyl)-5-(4-chlorophenyl)-pyrazole 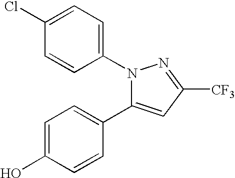
1-(4-chloroyphenyl)-3-(trifluoromethyl)-5-(4-hydroxyphenyl)-pyrazole 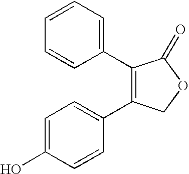
4-(4-hydroxyphenyl)-3-phenyl furan-2(5H)-one 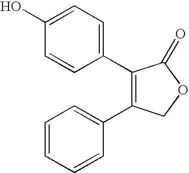
3-(4-hydroxyphenyl)-4-phenylfuran-2(5H)-one 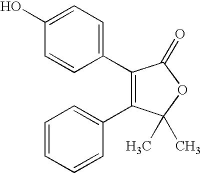
3-(4-hydroxyphenyl)-5,5-dimethyl-4-phenylfuran-2(5H)-one 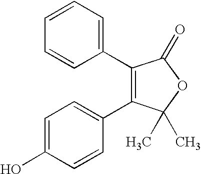
4-(4-hydroxyphenyl)-5,5-dimethyl-3-phenylfuran-2(5H)-one 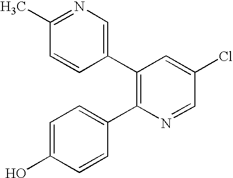
4-(5-chloro-6′-methyl-3,3′-bipyridin-2-yl)phenol 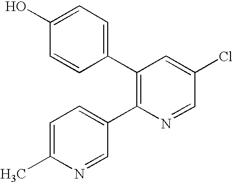
4-(5-chloro-6′-methyl-2,3′-bipyridin-3-yl)phenol 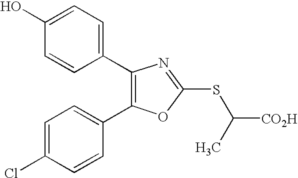
2-{[5-(4-chlorophenyl)-4-(4-hydroxyphenyl)-1,3-oxazol-2-yl]thio}propionic acid 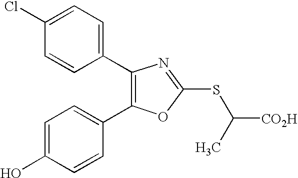
2-{[4-(4-chlorophenyl)-5-(4-hydroxyphenyl)-1,3-oxazol-2-yl]thio}propionic acid 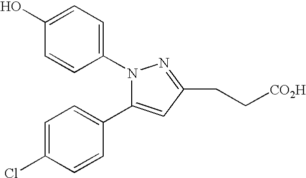
3-[5-(4-chlorophenyl)-1-(4-hydroxyphenyl)-1H-pyrazol-3-yl]propionic acid 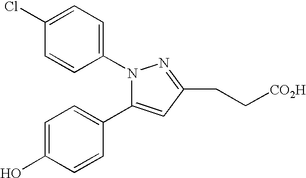
3-[1-(4-chlorophenyl)-5-(4-hydroxyphenyl)-1H-pyrazol-3-yl]propionic acid.

23. A pharmaceutical composition comprising the compound of claim 1 and a pharmaceutically acceptable carrier.

24-28. (canceled)

29. A method for treating inflammation, the method comprising providing a patient with a therapeutically effective serum concentration of the compound of claim 1.

30. (canceled)

31. A method for treating pain, the method comprising providing a patient with a therapeutically effective serum concentration of the compound of claim 1.

32-36. (canceled)

37. A method for treating anxiety comprising administering the compound of claim 1.

38-40. (canceled)

41. A method for treating a sleep disorder comprising administering the compound of claim 1.

42-44. (canceled)
